SEQUENCE LISTING (76 sequences)

SEQ ID NO: 1 - protein, 11 aa, Artificial Sequence
  This sequence is sysnthesized.
AQWPPKTTSAQ

SEQ ID NO: 2 - protein, 11 aa, Artificial Sequence
  This sequence is sysnthesized.
LMTPPKTTSAQ

SEQ ID NO: 3 - protein, 11 aa, Artificial Sequence
  This sequence is sysnthesized.
ATEPPKTTSAQ

SEQ ID NO: 4 - protein, 11 aa, Artificial Sequence
  This sequence is sysnthesized.
AQQRPPREPAQ

SEQ ID NO: 5 - protein, 11 aa, Artificial Sequence
  This sequence is sysnthesized.
AQQRPPRPADQ

SEQ ID NO: 6 - protein, 736 aa, Artificial Sequence
  This sequence is sysnthesized.
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 7 - protein, 743 aa, Artificial Sequence
  This sequence is sysnthesized.
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQWPPKTTSAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 8 - protein, 743 aa, Artificial Sequence
  This sequence is sysnthesized.
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQQRPPREPAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 9 - protein, 743 aa, Artificial Sequence
  This sequence is sysnthesized.
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQQRPPRPADQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 10 - protein, 743 aa, Artificial Sequence
  This sequence is sysnthesized.
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSLMTPPKTTSAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 11 - protein, 743 aa, Artificial Sequence
  This sequence is sysnthesized.
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSATEPPKTTSAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 12 - DNA, 36 bp, Artificial Sequence
  This sequence is sysnthesized.
actcatcgaccaatacttgtactatctctctagaac

SEQ ID NO: 13 - DNA, 83 bp, Artificial Sequence
  This sequence is sysnthesized.
  misc_feature (41)..(41): m is a or c
  misc_feature (42)..(43): n is a or g or c or t/u
  misc_feature (42)..(42): n is a, c, g, t or u
  misc_feature (43)..(43): n is a, c, g, t or u
  misc_feature (44)..(44): m is a or c
  misc_feature (45)..(46): n is a or g or c or t/u
  misc_feature (45)..(45): n is a, c, g, t or u
  misc_feature (46)..(46): n is a, c, g, t or u
  misc_feature (47)..(47): m is a or c
  misc_feature (48)..(49): n is a or g or c or t/u
  misc_feature (48)..(48): n is a, c, g, t or u
  misc_feature (49)..(49): n is a, c, g, t or u
  misc_feature (50)..(50): m is a or c
  misc_feature (51)..(52): n is a or g or c or t/u
  misc_feature (51)..(51): n is a, c, g, t or u
  misc_feature (52)..(52): n is a, c, g, t or u
  misc_feature (53)..(53): m is a or c
  misc_feature (54)..(55): n is a or g or c or t/u
  misc_feature (54)..(54): n is a, c, g, t or u
  misc_feature (55)..(55): n is a, c, g, t or u
  misc_feature (56)..(56): m is a or c
  misc_feature (57)..(58): n is a or g or c or t/u
  misc_feature (57)..(57): n is a, c, g, t or u
  misc_feature (58)..(58): n is a, c, g, t or u
  misc_feature (59)..(59): m is a or c
  misc_feature (60)..(61): n is a or g or c or t/u
  misc_feature (60)..(60): n is a, c, g, t or u
  misc_feature (61)..(61): n is a, c, g, t or u
gtattccttggttttgaacccaaccggtctgcgcctgtgcmnnmnnmnnmnnmnnmnnmnnttgggcactctggtggtttgtg

SEQ ID NO: 14 - DNA, 27 bp, Artificial Sequence
  This sequence is sysnthesized.
caggtcttcacggactcagactatcag

SEQ ID NO: 15 - DNA, 34 bp, Artificial Sequence
  This sequence is sysnthesized.
cattgatgagtttggacaaaccacaactagaatg

SEQ ID NO: 16 - DNA, 56 bp, Artificial Sequence
  This sequence is sysnthesized.
acactctttccctacacgacgctcttccgatctgacaagtggccacaaaccaccag

SEQ ID NO: 17 - DNA, 55 bp, Artificial Sequence
  This sequence is sysnthesized.
gtgactggagttcagacgtgtgctcttccgatctccttggttttgaacccaaccg

SEQ ID NO: 18 - DNA, 34 bp, Artificial Sequence
  This sequence is sysnthesized.
cattctagttgtggtttgtccaaactcatcaatg

SEQ ID NO: 19 - DNA, 25 bp, Artificial Sequence
  This sequence is sysnthesized.
aatagcttgatgaatcctggacctg

SEQ ID NO: 20 - DNA, 23 bp, Artificial Sequence
  This sequence is sysnthesized.
ttccttggttttgaacccaaccg

SEQ ID NO: 21 - DNA, 83 bp, Artificial Sequence
  This sequence is sysnthesized.
  misc_feature (59)..(59): m is a or c
  misc_feature (60)..(61): n is a or g or c or t/u
  misc_feature (60)..(60): n is a, c, g, t or u
  misc_feature (61)..(61): n is a, c, g, t or u
  misc_feature (62)..(62): m is a or c
  misc_feature (63)..(64): n is a or g or c or t/u
  misc_feature (63)..(63): n is a, c, g, t or u
  misc_feature (64)..(64): n is a, c, g, t or u
  misc_feature (65)..(65): m is a or c
  misc_feature (66)..(67): n is a or g or c or t/u
  misc_feature (66)..(66): n is a, c, g, t or u
  misc_feature (67)..(67): n is a, c, g, t or u
gtattccttggttttgaacccaaccggtctgcgcctgtgcagacgtagtcttcggcggmnnmnnmnnactctggtggtttgtg

SEQ ID NO: 22 - DNA, 83 bp, Artificial Sequence
  This sequence is sysnthesized.
  misc_feature (56)..(56): m is a or c
  misc_feature (57)..(59): n is a or g or c or t/u
  misc_feature (57)..(57): n is a, c, g, t or u
  misc_feature (58)..(58): n is a, c, g, t or u
  misc_feature (59)..(59): m is a or c
  misc_feature (60)..(61): n is a or g or c or t/u
  misc_feature (60)..(60): n is a, c, g, t or u
  misc_feature (61)..(61): n is a, c, g, t or u
  misc_feature (62)..(62): m is a or c
  misc_feature (63)..(64): n is a or g or c or t/u
  misc_feature (63)..(63): n is a, c, g, t or u
  misc_feature (64)..(64): n is a, c, g, t or u
gtattccttggttttgaacccaaccggtctgcgcctgtgcagacgtagtcttcggmnnmnnmnnggcactctggtggtttgtg

SEQ ID NO: 23 - DNA, 83 bp, Artificial Sequence
  This sequence is sysnthesized.
  misc_feature (53)..(53): m is a or c
  misc_feature (54)..(55): n is a or g or c or t/u
  misc_feature (54)..(54): n is a, c, g, t or u
  misc_feature (55)..(55): n is a, c, g, t or u
  misc_feature (56)..(56): m is a or c
  misc_feature (57)..(58): n is a or g or c or t/u
  misc_feature (57)..(57): n is a, c, g, t or u
  misc_feature (58)..(58): n is a, c, g, t or u
  misc_feature (59)..(59): m is a or c
  misc_feature (60)..(61): n is a or g or c or t/u
  misc_feature (60)..(60): n is a, c, g, t or u
  misc_feature (61)..(61): n is a, c, g, t or u
gtattccttggttttgaacccaaccggtctgcgcctgtgcagacgtagtcttmnnmnnmnnttgggcactctggtggtttgtg

SEQ ID NO: 24 - DNA, 83 bp, Artificial Sequence
  This sequence is sysnthesized.
  misc_feature (50)..(50): m is a or c
  misc_feature (51)..(52): n is a or g or c or t/u
  misc_feature (51)..(51): n is a, c, g, t or u
  misc_feature (52)..(52): n is a, c, g, t or u
  misc_feature (53)..(53): m is a or c
  misc_feature (54)..(55): n is a or g or c or t/u
  misc_feature (54)..(54): n is a, c, g, t or u
  misc_feature (55)..(55): n is a, c, g, t or u
  misc_feature (56)..(56): m is a or c
  misc_feature (57)..(58): n is a or g or c or t/u
  misc_feature (57)..(57): n is a, c, g, t or u
  misc_feature (58)..(58): n is a, c, g, t or u
gtattccttggttttgaacccaaccggtctgcgcctgtgcagacgtagtmnnmnnmnnccattgggcactctggtggtttgtg

SEQ ID NO: 25 - DNA, 83 bp, Artificial Sequence
  This sequence is sysnthesized.
  misc_feature (47)..(47): m is a or c
  misc_feature (48)..(49): n is a or g or c or t/u
  misc_feature (48)..(48): n is a, c, g, t or u
  misc_feature (49)..(49): n is a, c, g, t or u
  misc_feature (50)..(50): m is a or c
  misc_feature (51)..(52): n is a or g or c or t/u
  misc_feature (51)..(51): n is a, c, g, t or u
  misc_feature (52)..(52): n is a, c, g, t or u
  misc_feature (53)..(53): m is a or c
  misc_feature (54)..(55): n is a or g or c or t/u
  misc_feature (54)..(54): n is a, c, g, t or u
  misc_feature (55)..(55): n is a, c, g, t or u
gtattccttggttttgaacccaaccggtctgcgcctgtgcagacgtmnnmnnmnncggccattgggcactctggtggtttgtg

SEQ ID NO: 26 - DNA, 83 bp, Artificial Sequence
  This sequence is sysnthesized.
  misc_feature (44)..(44): m is a or c
  misc_feature (45)..(46): n is a or g or c or t/u
  misc_feature (45)..(45): n is a, c, g, t or u
  misc_feature (46)..(46): n is a, c, g, t or u
  misc_feature (47)..(47): m is a or c
  misc_feature (48)..(49): n is a or g or c or t/u
  misc_feature (48)..(48): n is a, c, g, t or u
  misc_feature (49)..(49): n is a, c, g, t or u
  misc_feature (50)..(50): m is a or c
  misc_feature (51)..(52): n is a or g or c or t/u
  misc_feature (51)..(51): n is a, c, g, t or u
  misc_feature (52)..(52): n is a, c, g, t or u
gtattccttggttttgaacccaaccggtctgcgcctgtgcagamnnmnnmnncggcggccattgggcactctggtggtttgtg

SEQ ID NO: 27 - DNA, 83 bp, Artificial Sequence
  This sequence is sysnthesized.
  misc_feature (41)..(41): m is a or c
  misc_feature (42)..(43): n is a or g or c or t/u
  misc_feature (42)..(42): n is a, c, g, t or u
  misc_feature (43)..(43): n is a, c, g, t or u
  misc_feature (44)..(44): m is a or c
  misc_feature (45)..(46): n is a or g or c or t/u
  misc_feature (45)..(45): n is a, c, g, t or u
  misc_feature (46)..(46): n is a, c, g, t or u
  misc_feature (47)..(47): m is a or c
  misc_feature (48)..(49): n is a or g or c or t/u
  misc_feature (48)..(48): n is a, c, g, t or u
  misc_feature (49)..(49): n is a, c, g, t or u
gtattccttggttttgaacccaaccggtctgcgcctgtgcmnnmnnmnncttcggcggccattgggcactctggtggtttgtg

SEQ ID NO: 28 - DNA, 83 bp, Artificial Sequence
  This sequence is sysnthesized.
  misc_feature (38)..(38): m is a or c
  misc_feature (39)..(40): n is a or g or c or t/u
  misc_feature (39)..(39): n is a, c, g, t or u
  misc_feature (40)..(40): n is a, c, g, t or u
  misc_feature (41)..(41): m is a or c
  misc_feature (42)..(43): n is a or g or c or t/u
  misc_feature (42)..(42): n is a, c, g, t or u
  misc_feature (43)..(43): n is a, c, g, t or u
  misc_feature (44)..(44): m is a or c
  misc_feature (45)..(46): n is a or g or c or t/u
  misc_feature (45)..(45): n is a, c, g, t or u
  misc_feature (46)..(46): n is a, c, g, t or u
gtattccttggttttgaacccaaccggtctgcgcctgmnnmnnmnnagtcttcggcggccattgggcactctggtggtttgtg

SEQ ID NO: 29 - DNA, 83 bp, Artificial Sequence
  This sequence is sysnthesized.
  misc_feature (35)..(35): m is a or c
  misc_feature (36)..(37): n is a or g or c or t/u
  misc_feature (36)..(36): n is a, c, g, t or u
  misc_feature (37)..(37): n is a, c, g, t or u
  misc_feature (38)..(38): m is a or c
  misc_feature (39)..(40): n is a or g or c or t/u
  misc_feature (39)..(39): n is a, c, g, t or u
  misc_feature (40)..(40): n is a, c, g, t or u
  misc_feature (41)..(41): m is a or c
  misc_feature (42)..(43): n is a or g or c or t/u
  misc_feature (42)..(42): n is a, c, g, t or u
  misc_feature (43)..(43): n is a, c, g, t or u
gtattccttggttttgaacccaaccggtctgcgcmnnmnnmnncgtagtcttcggcggccattgggcactctggtggtttgtg

SEQ ID NO: 30 - DNA, 83 bp, Artificial Sequence
  This sequence is sysnthesized.
  misc_feature (35)..(35): m is a or c
  misc_feature (36)..(37): n is a or g or c or t/u
  misc_feature (36)..(36): n is a, c, g, t or u
  misc_feature (37)..(37): n is a, c, g, t or u
  misc_feature (38)..(38): m is a or c
  misc_feature (39)..(40): n is a or g or c or t/u
  misc_feature (39)..(39): n is a, c, g, t or u
  misc_feature (40)..(40): n is a, c, g, t or u
  misc_feature (62)..(62): m is a or c
  misc_feature (63)..(64): n is a or g or c or t/u
  misc_feature (63)..(63): n is a, c, g, t or u
  misc_feature (64)..(64): n is a, c, g, t or u
  misc_feature (65)..(65): m is a or c
  misc_feature (66)..(67): n is a or g or c or t/u
  misc_feature (66)..(66): n is a, c, g, t or u
  misc_feature (67)..(67): n is a, c, g, t or u
gtattccttggttttgaacccaaccggtctgcgcmnnmnnagacgtagtcttcggcggccamnnmnnactctggtggtttgtg

SEQ ID NO: 31 - DNA, 83 bp, Artificial Sequence
  This sequence is sysnthesized.
  misc_feature (59)..(59): m is a or c
  misc_feature (60)..(61): n is a or g or c or t/u
  misc_feature (60)..(60): n is a, c, g, t or u
  misc_feature (61)..(61): n is a, c, g, t or u
  misc_feature (62)..(62): m is a or c
  misc_feature (63)..(64): n is a or g or c or t/u
  misc_feature (63)..(63): n is a, c, g, t or u
  misc_feature (64)..(64): n is a, c, g, t or u
  misc_feature (65)..(65): m is a or c
  misc_feature (66)..(67): n is a or g or c or t/u
  misc_feature (66)..(66): n is a, c, g, t or u
  misc_feature (67)..(67): n is a, c, g, t or u
gtattccttggttttgaacccaaccggtctgcgcctgtgccggctcacgcggaggcctmnnmnnmnnactctggtggtttgtg

SEQ ID NO: 32 - DNA, 83 bp, Artificial Sequence
  This sequence is sysnthesized.
  misc_feature (56)..(56): m is a or c
  misc_feature (57)..(58): n is a or g or c or t/u
  misc_feature (57)..(57): n is a, c, g, t or u
  misc_feature (58)..(58): n is a, c, g, t or u
  misc_feature (59)..(59): m is a or c
  misc_feature (60)..(61): n is a or g or c or t/u
  misc_feature (60)..(60): n is a, c, g, t or u
  misc_feature (61)..(61): n is a, c, g, t or u
  misc_feature (62)..(62): m is a or c
  misc_feature (63)..(64): n is a or g or c or t/u
  misc_feature (63)..(63): n is a, c, g, t or u
  misc_feature (64)..(64): n is a, c, g, t or u
gtattccttggttttgaacccaaccggtctgcgcctgtgccggctcacgcggaggmnnmnnmnnggcactctggtggtttgtg

SEQ ID NO: 33 - DNA, 83 bp, Artificial Sequence
  This sequence is sysnthesized.
  misc_feature (53)..(53): m is a or c
  misc_feature (54)..(55): n is a or g or c or t/u
  misc_feature (54)..(54): n is a, c, g, t or u
  misc_feature (55)..(55): n is a, c, g, t or u
  misc_feature (56)..(56): m is a or c
  misc_feature (57)..(58): n is a or g or c or t/u
  misc_feature (57)..(57): n is a, c, g, t or u
  misc_feature (58)..(58): n is a, c, g, t or u
  misc_feature (59)..(59): m is a or c
  misc_feature (60)..(61): n is a or g or c or t/u
  misc_feature (60)..(60): n is a, c, g, t or u
  misc_feature (61)..(61): n is a, c, g, t or u
gtattccttggttttgaacccaaccggtctgcgcctgtgccggctcacgcggmnnmnnmnnttgggcactctggtggtttgtg

SEQ ID NO: 34 - DNA, 83 bp, Artificial Sequence
  This sequence is sysnthesized.
  misc_feature (50)..(50): m is a or c
  misc_feature (51)..(52): n is a or g or c or t/u
  misc_feature (51)..(51): n is a, c, g, t or u
  misc_feature (52)..(52): n is a, c, g, t or u
  misc_feature (53)..(53): m is a or c
  misc_feature (54)..(55): n is a or g or c or t/u
  misc_feature (54)..(54): n is a, c, g, t or u
  misc_feature (55)..(55): n is a, c, g, t or u
  misc_feature (56)..(56): m is a or c
  misc_feature (57)..(58): n is a or g or c or t/u
  misc_feature (57)..(57): n is a, c, g, t or u
  misc_feature (58)..(58): n is a, c, g, t or u
gtattccttggttttgaacccaaccggtctgcgcctgtgccggctcacgmnnmnnmnnctgttgggcactctggtggtttgtg

SEQ ID NO: 35 - DNA, 83 bp, Artificial Sequence
  This sequence is sysnthesized.
  misc_feature (47)..(47): m is a or c
  misc_feature (48)..(49): n is a or g or c or t/u
  misc_feature (48)..(48): n is a, c, g, t or u
  misc_feature (49)..(49): n is a, c, g, t or u
  misc_feature (50)..(50): m is a or c
  misc_feature (51)..(52): n is a or g or c or t/u
  misc_feature (51)..(51): n is a, c, g, t or u
  misc_feature (52)..(52): n is a, c, g, t or u
  misc_feature (53)..(53): m is a or c
  misc_feature (54)..(55): n is a or g or c or t/u
  misc_feature (54)..(54): n is a, c, g, t or u
  misc_feature (55)..(55): n is a, c, g, t or u
gtattccttggttttgaacccaaccggtctgcgcctgtgccggctcmnnmnnmnncctctgttgggcactctggtggtttgtg

SEQ ID NO: 36 - DNA, 83 bp, Artificial Sequence
  This sequence is sysnthesized.
  misc_feature (44)..(44): m is a or c
  misc_feature (45)..(46): n is a or g or c or t/u
  misc_feature (45)..(45): n is a, c, g, t or u
  misc_feature (46)..(46): n is a, c, g, t or u
  misc_feature (47)..(47): m is a or c
  misc_feature (48)..(49): n is a or g or c or t/u
  misc_feature (48)..(48): n is a, c, g, t or u
  misc_feature (49)..(49): n is a, c, g, t or u
  misc_feature (50)..(50): m is a or c
  misc_feature (51)..(52): n is a or g or c or t/u
  misc_feature (51)..(51): n is a, c, g, t or u
  misc_feature (52)..(52): n is a, c, g, t or u
gtattccttggttttgaacccaaccggtctgcgcctgtgccggmnnmnnmnnaggcctctgttgggcactctggtggtttgtg

SEQ ID NO: 37 - DNA, 83 bp, Artificial Sequence
  This sequence is sysnthesized.
  misc_feature (41)..(41): m is a or c
  misc_feature (42)..(43): n is a or g or c or t/u
  misc_feature (42)..(42): n is a, c, g, t or u
  misc_feature (43)..(43): n is a, c, g, t or u
  misc_feature (44)..(44): m is a or c
  misc_feature (45)..(46): n is a or g or c or t/u
  misc_feature (45)..(45): n is a, c, g, t or u
  misc_feature (46)..(46): n is a, c, g, t or u
  misc_feature (47)..(47): m is a or c
  misc_feature (48)..(49): n is a or g or c or t/u
  misc_feature (48)..(48): n is a, c, g, t or u
  misc_feature (49)..(49): n is a, c, g, t or u
gtattccttggttttgaacccaaccggtctgcgcctgtgcmnnmnnmnncggaggcctctgttgggcactctggtggtttgtg

SEQ ID NO: 38 - DNA, 83 bp, Artificial Sequence
  This sequence is sysnthesized.
  misc_feature (38)..(38): m is a or c
  misc_feature (39)..(40): n is a or g or c or t/u
  misc_feature (39)..(39): n is a, c, g, t or u
  misc_feature (40)..(40): n is a, c, g, t or u
  misc_feature (41)..(41): m is a or c
  misc_feature (42)..(43): n is a or g or c or t/u
  misc_feature (42)..(42): n is a, c, g, t or u
  misc_feature (43)..(43): n is a, c, g, t or u
  misc_feature (44)..(44): m is a or c
  misc_feature (45)..(46): n is a or g or c or t/u
  misc_feature (45)..(45): n is a, c, g, t or u
  misc_feature (46)..(46): n is a, c, g, t or u
gtattccttggttttgaacccaaccggtctgcgcctgmnnmnnmnnacgcggaggcctctgttgggcactctggtggtttgtg

SEQ ID NO: 39 - DNA, 83 bp, Artificial Sequence
  This sequence is sysnthesized.
  misc_feature (35)..(35): m is a or c
  misc_feature (36)..(37): n is a or g or c or t/u
  misc_feature (36)..(36): n is a, c, g, t or u
  misc_feature (37)..(37): n is a, c, g, t or u
  misc_feature (38)..(38): m is a or c
  misc_feature (39)..(40): n is a or g or c or t/u
  misc_feature (39)..(39): n is a, c, g, t or u
  misc_feature (40)..(40): n is a, c, g, t or u
  misc_feature (41)..(41): m is a or c
  misc_feature (42)..(43): n is a or g or c or t/u
  misc_feature (42)..(42): n is a, c, g, t or u
  misc_feature (43)..(43): n is a, c, g, t or u
gtattccttggttttgaacccaaccggtctgcgcmnnmnnmnnctcacgcggaggcctctgttgggcactctggtggtttgtg

SEQ ID NO: 40 - DNA, 83 bp, Artificial Sequence
  This sequence is sysnthesized.
  misc_feature (35)..(35): m is a or c
  misc_feature (36)..(37): n is a or g or c or t/u
  misc_feature (36)..(36): n is a, c, g, t or u
  misc_feature (37)..(37): n is a, c, g, t or u
  misc_feature (38)..(38): m is a or c
  misc_feature (39)..(40): n is a or g or c or t/u
  misc_feature (39)..(39): n is a, c, g, t or u
  misc_feature (40)..(40): n is a, c, g, t or u
  misc_feature (62)..(62): m is a or c
  misc_feature (63)..(64): n is a or g or c or t/u
  misc_feature (63)..(63): n is a, c, g, t or u
  misc_feature (64)..(64): n is a, c, g, t or u
  misc_feature (65)..(65): m is a or c
  misc_feature (66)..(67): n is a or g or c or t/u
  misc_feature (66)..(66): n is a, c, g, t or u
  misc_feature (67)..(67): n is a, c, g, t or u
gtattccttggttttgaacccaaccggtctgcgcmnnmnncggctcacgcggaggcctctgmnnmnnactctggtggtttgtg

SEQ ID NO: 41 - protein, 599 aa, Artificial Sequence
  This sequence is sysnthesized.
TAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 42 - protein, 534 aa, Artificial Sequence
  This sequence is sysnthesized.
MASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 43 - protein, 606 aa, Artificial Sequence
  This sequence is sysnthesized.
TAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQWPPKTTSAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 44 - protein, 541 aa, Artificial Sequence
  This sequence is sysnthesized.
MASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQWPPKTTSAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 45 - protein, 606 aa, Artificial Sequence
  This sequence is sysnthesized.
TAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQQRPPREPAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 46 - protein, 541 aa, Artificial Sequence
  This sequence is sysnthesized.
MASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQQRPPREPAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 47 - protein, 606 aa, Artificial Sequence
  This sequence is sysnthesized.
TAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQQRPPRPADQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 48 - protein, 541 aa, Artificial Sequence
  This sequence is sysnthesized.
MASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQQRPPRPADQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 49 - protein, 606 aa, Artificial Sequence
  This sequence is sysnthesized.
TAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSLMTPPKTTSAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 50 - protein, 541 aa, Artificial Sequence
  This sequence is sysnthesized.
MASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSLMTPPKTTSAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 51 - protein, 606 aa, Artificial Sequence
  This sequence is sysnthesized.
TAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSATEPPKTTSAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 52 - protein, 541 aa, Artificial Sequence
  This sequence is sysnthesized.
MASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSATEPPKTTSAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 53 - protein, 743 aa, Artificial Sequence
  This sequence is sysnthesized.
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQTAFPREPAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 54 - protein, 606 aa, Artificial Sequence
  This sequence is sysnthesized.
TAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQTAFPREPAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 55 - protein, 541 aa, Artificial Sequence
  This sequence is sysnthesized.
MASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQTAFPREPAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 56 - protein, 743 aa, Artificial Sequence
  This sequence is sysnthesized.
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAPARPPREPAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 57 - protein, 606 aa, Artificial Sequence
  This sequence is sysnthesized.
TAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAPARPPREPAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 58 - protein, 541 aa, Artificial Sequence
  This sequence is sysnthesized.
MASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAPARPPREPAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 59 - protein, 743 aa, Artificial Sequence
  This sequence is sysnthesized.
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQPTSKTTSAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 60 - protein, 606 aa, Artificial Sequence
  This sequence is sysnthesized.
TAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQPTSKTTSAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 61 - protein, 541 aa, Artificial Sequence
  This sequence is sysnthesized.
MASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQPTSKTTSAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 62 - protein, 743 aa, Artificial Sequence
  This sequence is sysnthesized.
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQLMVKTTSAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 63 - protein, 606 aa, Artificial Sequence
  This sequence is sysnthesized.
TAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQLMVKTTSAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 64 - protein, 541 aa, Artificial Sequence
  This sequence is sysnthesized.
MASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQLMVKTTSAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 65 - protein, 743 aa, Artificial Sequence
  This sequence is sysnthesized.
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQWTDKTTSAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 66 - protein, 606 aa, Artificial Sequence
  This sequence is sysnthesized.
TAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQWTDKTTSAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 67 - protein, 541 aa, Artificial Sequence
  This sequence is sysnthesized.
MASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQWTDKTTSAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 68 - protein, 743 aa, Artificial Sequence
  This sequence is sysnthesized.
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAVLSPKTTSAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 69 - protein, 606 aa, Artificial Sequence
  This sequence is sysnthesized.
TAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAVLSPKTTSAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 70 - protein, 541 aa, Artificial Sequence
  This sequence is sysnthesized.
MASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAVLSPKTTSAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 71 - protein, 11 aa, Artificial Sequence
  This sequence is sysnthesized.
AQPTSKTTSAQ

SEQ ID NO: 72 - protein, 11 aa, Artificial Sequence
  This sequence is sysnthesized.
AQLMVKTTSAQ

SEQ ID NO: 73 - protein, 11 aa, Artificial Sequence
  This sequence is sysnthesized.
AQWTDKTTSAQ

SEQ ID NO: 74 - protein, 11 aa, Artificial Sequence
  This sequence is sysnthesized.
AVLSPKTTSAQ

SEQ ID NO: 75 - protein, 11 aa, Artificial Sequence
  This sequence is sysnthesized.
AQTAFPREPAQ

SEQ ID NO: 76 - protein, 11 aa, Artificial Sequence
  This sequence is sysnthesized.
APARPPREPAQ

(19) United States
(12) Patent Application Publication
(10) Pub. No.: US 2025/0297279 A1
(43) Pub. Date: Sep. 25, 2025

(54) RECOMBINANT AAV VECTORS AND USE THEREOF

(71) Applicant: GENANS BIOTECHNOLOGY CO., LTD, Beijing (CN)

(72) Inventors: Rui Lin, Beijing (CN); Minmin Luo, Beijing (CN)

(21) Appl. No.: 18/720,955

(22) PCT Filed: Dec. 15, 2021
(86) PCT No.: PCT/CN2021/138535

§ 371 Date: Jun. 17, 2024

(51) Int. Cl. C12N 15/86; C07K 14/005

(52) U.S. Cl. CPC C12N 15/86; C07K 14/005; C12N 2750/14122; C12N 2750/14143

(57) ABSTRACT

Provided is a recombinant adeno-associated virus (rAAV) capsid protein, comprising an amino acid sequence of 11 contiguous amino acids X₁X₂X₃X₄X₅X₆X₇X₈X₉X₁₀Q, rAAV vector comprising the same, and the use thereof.

### REFERENCE TO SEQUENCE LISTING

Applicant submits herewith a Sequence Listing in computer readable form and in compliance with 37 C.F.R. §§ 1.821-1.825. Please amend the specification to direct entry of the attached sequence listing in ASCII TXT format entitled WO11393BSUS-Sequence-listing.txt. This ASCII text file is 228,545 bytes in size and was created on Dec. 30, 2024.

### FIELD OF THE INVENTION

The present disclosure relates to the field of adeno-associated virus (AAV) vectors, particularly to recombinant AAV (rAAV) capsid proteins, rAAV vectors containing the rAAV capsid proteins and the use thereof.

### BACKGROUND

Microglia account for about 10% of the total cell population in the central nervous system (CNS). Originally viewed as debris scavengers, microglia are now considered as the major regulator of the CNS under both normal and pathological conditions. Microglia conduct active surveillance, and initiate rapid innate and adaptive immune responses upon encountering immune assaults. Beyond their functions in immunity, recent studies have revealed multifaceted roles of microglia in controlling neural circuit development and plasticity. Emerging evidences have recognized microglial dysfunction as a key factor in CNS ageing and in the progression of CNS diseases including neurological disorders and brain cancers. Many clinical studies have identified risk-associated alterations in genes that are highly expressed by microglia, highlighting the engagement of microglia in CNS disease progression and the potential for targeting microglia for therapeutic interventions.

A key challenge in studying microglia lies in the incapability to efficiently label and manipulate them. Current approaches rely heavily on generating germline transgenic mouse models to introduce transgenes or genetic modifications into microglia, which is time-consuming, laborious, and often inefficient. Moreover, the involvement of germline transgenesis prevents its wide applications in animal models with low reproduction rate and long generation time (e.g., non-human primates), and excludes their uses as therapeutic tools in humans.

Recombinant viral vectors represent an attractive alternative for manipulating microglia, and hold great promises for microglial gene therapies. In particular, owing to a lack of obvious pathogenicity, recombinant adeno-associated viruses (rAAVs) are now the most frequently used viral vectors in basic research and in gene therapies. However, the transduction of microglia by rAAVs remains extremely poor, despite their ability to transduce a wide range of cell types in mammals. rAAVs packaged using existing AAV capsids have not achieved a high transduction rate and a sufficient transgene expression level in microglia, especially in vivo.

On the other hand, viral transduction of microglia (and macrophages in general) also faces the potential issue of inducing immune activation. As an example, recombinant adenoviruses efficiently transduce macrophages but at the same time make the transduced cells immune reactive.

Therefore, there is an unmet need to develop a system for achieving a high transduction rate and a sufficient transgene expression level in microglia, without triggering the immune reactivation of the transduced cells.

### SUMMARY OF THE INVENTION

To overcome at least one of the above technical problems, the present disclosure provides new AAV capsids that mediate efficient gene delivery to microglia, as well as the application thereof.

Provided herein are recombinant adeno-associated virus (rAAV) capsid proteins having a seven-amino-acid peptide insertion, as compared to a parental AAV capsid protein. When the rAAV capsid proteins provided herein are present in an AAV vector/virion, confer increased transduction efficiency of microglia, both in vivo and in vitro, as compared to an AAV virion without the peptide insertion. Also provided are recombinant AAV vectors/virions and pharmaceutical compositions thereof comprising the rAAV capsid proteins as provided therein; and methods for using these rAAV capsid proteins and vectors/virions in research and in clinical practice, for example, in the delivery of polynucleotide sequences to microglia for the treatment of diseases associated with microglia.

According to one aspect of the present disclosure, provided is a recombinant adeno-associated virus (rAAV) capsid protein, which comprises an amino acid sequence of 11 contiguous amino acids X₁X₂X₃X₄X₅X₆X₇X₈X₉X₁₀Q, wherein, X₁ is selected from Ala or Leu; X₂ is selected from Gln, Met, Thr, Val or Pro; X₃ is selected from Trp, Thr, Glu, Pro, Leu, Ala or Gln; X₄ is selected from Pro, Thr, Met, Ser, Arg or Ala; X₅ is selected from Pro, Ser, Val, Asp or Phe; X₆ is selected from Lys or Pro; X₇ is selected from Thr or Arg; X₈ is selected from Thr, Glu or Pro; X₉ is selected from Ser, Pro or Ala; and X₁₀ is selected from Ala or Asp.

In some embodiments, the rAAV capsid protein comprises an amino acid sequence of 11 contiguous amino acids X₁X₂X₃X₄X₅X₆X₇X₈X₉X₁₀Q, in which X₆ is Lys; X₇ is Thr; X₈ is Thr; X₉ is Ser; and/or X₁₀ is Ala. In some embodiments, the rAAV capsid protein comprises an amino acid sequence of 11 contiguous amino acids X₁X₂X₃X₄X₅KTTSAQ. In further embodiments, X₁ is selected from Ala or Leu. In further embodiments, X₂ is selected from Gln, Met, Thr or Val. In further embodiments, X₃ is selected from Trp, Thr, Glu, Pro or Leu. In further embodiments, X₄ is selected from Pro, Thr, Met or Ser. In further embodiments, X₅ is selected from Ser, Val, Asp or Pro. In some specific embodiments, X₁ is Ala, X₂ is Gln, X₃ is Trp, X₄ is Pro, and X₅ is Pro. In some specific embodiments, X₁ is Leu, X₂ is Met, X₃ is Thr, X₄ is Pro, and X₅ is Pro. In some specific embodiments, X₁ is Ala, X₂ is Thr; X₃ is Glu, X₄ is Pro, and X₅ is Pro. In some specific embodiments, X₁ is Ala, X₂ is Gln; X₃ is Pro, X₄ is Thr, and X₅ is Ser. In some specific embodiments, X₁ is Ala, X₂ is Gln; X₃ is Leu, X₄ is Met, and X₅ is Val. In some specific embodiments, X₁ is Ala, X₂ is Gln; X₃ is Trp, X₄ is Thr, and X₅ is Asp. In some specific embodiments, X₁ is Ala, X₂ is Val; X₃ is Leu, X₄ is Ser, and X₅ is Pro.

In some specific embodiments, the rAAV capsid protein comprises an amino acid sequence of AQWPPKTTSAQ (SEQ ID NO.: 1). In some specific embodiments, the rAAV capsid protein comprises an amino acid sequence of LMTPPKTTSAQ (SEQ ID NO.: 2). In some specific embodiments, the rAAV capsid protein comprises an amino acid sequence of ATEPPKTTSAQ (SEQ ID NO.: 3). In some specific embodiments, the rAAV capsid protein comprises an amino acid sequence of AQPTSKTTSAQ (SEQ ID NO.: 71). In some specific embodiments, the rAAV capsid protein comprises an amino acid sequence of AQLMVKTTSAQ (SEQ ID NO.: 72). In some specific embodiments, the rAAV capsid protein comprises an amino acid sequence of AQWTDKTTSAQ (SEQ ID NO.: 73). In some specific embodiments, the rAAV capsid protein comprises an amino acid sequence of AVLSPKTTSAQ (SEQ ID NO.: 74).

In some embodiments, the rAAV capsid protein, comprises an amino acid sequence X₁X₂X₃X₄X₅X₆X₇X₈X₉X₁₀Q, wherein, X₁ is Ala; X₆ is Pro; and/or X₇ is Arg. In some embodiments, the rAAV capsid protein comprises an amino acid sequence AX₂X₃X₄X₅PRX₈X₉X₁₀Q. In further embodiments, X₂ is selected from Gln or Pro. In further embodiments, X₃ is selected from Thr, Ala or Gln. In further embodiments, X₄ is selected from Arg or Ala. In further embodiments, X₅ is selected from Pro or Phe. In further embodiments, X₈ is selected from Glu or Pro. In further embodiments, X₉ is selected from Pro or Ala. In further embodiments, X₁₀ is selected from Ala or Asp. In some specific embodiments, X₂ is Gln, X₃ is Gln, X₄ is Arg, Xₛ is Pro, Xₛ is Glu, X₉ is Pro, and X₁₀ is Ala. In some specific embodiments, X₂ is Gln, X₃ is Gln, X₄ is Arg, Xₛ is Pro, Xₛ is Pro, X₉ is Ala, and X₁₀ is Asp. In some specific embodiments, X₂ is Gln, X₃ is Thr, X₄ is Ala, X₅ is Phe, X₈ is Glu, X₉ is Pro, and X₁₀ is Ala. In some specific embodiments, X₂ is Pro, X₃ is Ala, X₄ is Arg, X₅ is Pro, X₈ is Glu, X₉ is Pro, and X₁₀ is Ala.

In some specific embodiments, the rAAV capsid protein comprises an amino acid sequence of AQQRPPREPAQ (SEQ ID NO.: 4). In some specific embodiments, the rAAV capsid protein comprises an amino acid sequence of AQQRPPRPADQ (SEQ ID NO.: 5). In some specific embodiments, the rAAV capsid protein comprises an amino acid sequence ofAQTAFPREPAQ (SEQ ID NO.: 75). In some specific embodiments, the rAAV capsid protein comprises an amino acid sequence of APARPPREPAQ (SEQ ID NO.: 76).

According to another aspect of the present disclosure, provided is a polynucleotide sequence which encodes the rAAV capsid protein provided by the present disclosure.

According to yet another aspect of the present disclosure, provided is a recombinant adeno-associated virus (rAAV) vector which comprises the capsid protein provided by the present disclosure. In some embodiments, the rAAV vector further comprises a heterologous polynucleotide sequence. In some embodiments, heterologous polynucleotide sequence encodes a heterologous polypeptide, a non-coding RNA or a CRISPR agent.

In some embodiments, the CRISPR agent comprises a DNA-targeting RNA, e.g., a crRNA-like RNA, a tracrRNA-like RNA, a single guide RNA, and the like. In some embodiments, the heterologous polynucleotide sequence encodes a protein, such as antibody, membrane protein (e.g. a receptor), chaperone, or ubiquitin ligase. In some embodiments, the heterologous polynucleotide sequence encodes miRNA, siRNA, piRNA, lncRNA, or a guide RNA.

According to yet another aspect of the present disclosure, provided is a pharmaceutical composition comprising the rAAV vector/virion provided by the present disclosure, and a pharmaceutically acceptable carrier.

According to yet another aspect of the present disclosure, provided is a method for delivering the rAAV vector provided by the present disclosure to a target cell, which comprises contacting the target cell with the rAAV vector/virion. In some embodiments, the target cell is a microglia in vitro or in vivo. In some embodiments, the contacting is performed in the presence of an inhibitor for a topoisomerase or proteasome. In a certain embodiments, the contacting is performed in the presence of an inhibitor for a topoisomerase and/or a DNA damage inducer. The inhibitor for a topoisomerase may be selected from doxorubicin (a DNA topoisomerase II inhibitor), bortezomib (a proteasome inhibitor), etoposide (a DNA topoisomerase II inhibitor), teniposide (a DNA topoisomerase II inhibitor), vanillin (an inhibitor of non-homologous end joining) and the like. The DNA damage inducer may be bleomycin and the like.

According to yet another aspect of the present disclosure, provided is a host cell which comprises the polynucleotide sequence encoding the rAAV capsid protein provided by the present disclosure.

According to yet another aspect of the present disclosure, provided is a method for treating a neurological disorder, which comprises administering a therapeutically effective amount of the pharmaceutical composition to a subject in need thereof. In some embodiments, the neurological disorder may be a disease associated with microglia. In some embodiments, the neurological disorder may comprise Alzheimer's disease, Parkinson's disease, Huntington's disease and amyotrophic lateral sclerosis, leukoencephalopathy, glioma and atypical teratoid/rhabdoid tumor.

The development of engineered AAV capsids that are capable of mediating efficient microglial transduction provides much-needed viral tools for interrogating microglia biology. This disclosure demonstrated that the rAAVs provided herein enable sufficient transgene expression in microglia for labeling, monitoring, and manipulation. The newly evolved AAV capsids herein can facilitate applications of diverse genetically-encoded tools (e.g., fluorescent sensors for signaling molecules, and optogenetic and chemogenetic effectors) and gene editing methods in microglia both in vitro and in vivo. Combining the rAAV provided herein with other rAAVs targeting additional cell types in the CNS also represents a promising strategy to study the interactions between microglia and different cell types in the same animal. Recent single-cell transcriptomics studies have unveiled unexpectedly large regional heterogeneity of microglia in the brain. Thus, the rAAV of the present disclosure could be ideal tools for brain-region-specific microglia manipulation in vivo to investigate the roles of microglia in controlling neural circuits in different brain areas.

Genetics studies in human patients identified many druggable targets and signaling pathways of CNS diseases that are highly enriched in microglia. Introducing disease-related mutations into mouse models results in microglial dysfunction, and induces pathologies and behavioral phenotypes that resemble those in human patients, underscoring the great potential of microglia-based gene therapies. As a key premise for gene therapy, the efficient transfer of therapeutic reagents into target cells requires suitable vehicles. The lack of safe, efficient, and clinically-relevant delivery modalities has hindered the development of microglial gene therapies. The success in evolving AAV capsid of the present disclosure demonstrates the possibility of effective microglial transduction using rAAVs and lays the foundation for future optimization of AAV capsids for microglia-based gene therapies.

In other embodiments, the AAV vector/virion comprising the variant capsid protein in the preceding paragraphs may incorporate any of the preceding or subsequently disclosed embodiments. Indeed, it is appreciated that certain features disclosed herein, which are, for clarity, described in the context of separate embodiments, may also be provided in combination in a single embodiment. Conversely, various features disclosed herein, which are, for brevity, described in the context of a single embodiment, may also be provided separately or in any suitable sub-combination. All combinations of the embodiments pertaining to the invention are specifically embraced by the invention and are disclosed herein just as if each and every combination was individually and explicitly disclosed. In addition, all subcombinations of the various embodiments and elements thereof are also specifically embraced by the invention and are disclosed herein just as if each and every such subcombination was individually and explicitly disclosed herein.

### DESCRIPTION OF THE FIGURES

FIG. 1. Directed evolution generates AAV-MGs that mediate efficient microglial transduction in vitro and in vivo. (A) Schematic of the in vitro selection process. Random heptamers were inserted between the 588 and 589 amino acids of the AAV9 VP1 protein. The library was screened in cultured mouse microglia for two rounds. (B) Distributions of AAV9 capsid variants recovered from cultured mouse microglia, sorted by decreasing order of the enrichment score. The pie chart shows the normalized frequency of AAV-cMG.WPP among total recovered sequences. Cyan: AAV-cMG.WPP. (C) Schematic of the in vivo selection process. Semi-random mutations were introduced into the inserted heptamer and the adjacent four amino acids in AAV-cMG.WPP. The library was screened in the striatum and the midbrain of Cx3cr1^{CreER} mice. Capsid variants from Cre-recombined AAV genomes were selectively recovered from the brains. (D) Distributions of AAV-cMG.WPP variants recovered from the Cx3cr1^{CreER} mouse brains, sorted by decreasing order of the enrichment score. The pie chart shows the normalized frequency of AAV-MG1.1 and AAV-MG1.2 among total recovered sequences. Magenta: AAV-MG1.1, green: AAV-MG1.2, cyan: AAV-cMG.WPP. (E) Representative images showing the mScarlet expression patterns in the orbitofrontal cortex (OFC, top) and the striatum (bottom) of Cx3cr1^{CreER} mice injected with AAV-cMG.WPP-SFFV-DIO-mScarlet, AAV-MG1.1-SFFV-DIO-mScarlet, or AAV-MG1.2-SFFV-DIO-mScarlet. The AAVs were injected with or without retro-orbital administration of doxorubicin. The detailed quantification is shown in FIG. 5A. (F) Representative images showing the colocalization of mScarlet and Iba⁺ immunosignals in the OFC and the striatum of Cx3cr1^{CreER} mice injected with AAV-MG1.1-SFFV-DIO-mScarlet. The detailed quantification is shown in FIG. 5D. Scale bars, 250 μm (E), 100 μm (F).

FIG. 2. AAV-cMG mediates efficient microglia transduction in vitro. (A) Schematic of the selection process. Random seven amino acids were inserted between the 588 and 589 amino acids of AAV9 VP1 protein. The library was screened in cultured mouse microglia for two rounds. (B) Distributions of AAV9 capsid variants recovered from cultured mouse microglia, sorted by decreasing order of the enrichment score. The pie chart shows the normalized frequency of AAV-cMG.QRP in total recovered sequences. (C) Representative images of cultured mouse microglia transduced with mScarlet reporter AAVs packaged using AAV-MG.QRP. (D) Schematic of the selection process of AAV-MG.QRP variants. The right panel shows distributions of AAV-MG.QRP variants recovered from cultured mouse microglia, sorted by decreasing order of the enrichment score. The pie chart shows the normalized frequency of AAV-cMG in total recovered sequences. (E) Representative images of cultured mouse microglia transduced with mScarlet reporter AAVs packaged using different capsids. (F) Representative immunofluorescence images showing the colocalization of mScarlet and Iba⁺ immunosignals in cultured mouse microglia transduced with AAV-cMG-SFFV-mScarlet. (G) Quantification of mScarlet+percentage and the mean fluorescent intensity of cultured mouse microglia transduced with mScarlet reporter AAVs packaged using different capsids (n=6 replicates for each group in 3pt; 5 replicates for each group in 5pt; the bar represents the mean value for each group; one-way ANOVA with Dunnett's post-hoc test). (H) Representative images showing the cultured microglia transduced by AAV-cMG-SFFV-mScarlet with (right) or without (left) doxorubicin. (I) Quantification of mScarlet⁺ percentage and the mean fluorescent intensity of cultured mouse microglia transduced by AAV-cMG-SFFV-mScarlet with or without doxorubicin (n=6 replicates for each group; the bar represents the mean value for each group; one-way ANOVA with Dunnett's post-hoc test). Scale bars, 200 μm (C, E, H), 50 μm (F).

FIG. 3. Transduction of cultured mouse microglia by AAV-cMG.WPP. (A) Representative images of cultured mouse microglia transduced with mScarlet reporter rAAVs packaged using different capsids. (B) Representative immunofluorescent images showing the colocalization of mScarlet and Iba⁺ immunosignals in cultured mouse microglia transduced with mScarlet reporter rAAVs packaged using AAV-cMG.WPP. (C) Quantification of the mScarlet⁺ percentage and the mean fluorescent intensity of cultured mouse microglia transduced with mScarlet reporter rAAVs packaged using different capsids (n=4 replicates for each group; the bar represents the mean value for each group; one-way ANOVA with Dunnett's post-hoc test). (D) Principal component analysis of the transcriptomes of cultured mouse microglia from four treatment groups: control untransduced (homeostatic state), LPS-treated (reactive state), interleukin-4-treated (IL4; alternative activation state), and AAV-cMG.WPP-transduced group (n=3 replicates for each group). (E) A heatmap showing the expression of marker genes for microglial reactive and alternative activation states in different treatment groups as shown in (D). Scale bars, 100 μm (A), 50 μm (B).

FIG. 4. AAV-MGs mediate efficient microglial transduction in vivo. (A) Representative images showing the mScarlet expression patterns in the midbrain of Cx3cr1^{CreER} mice injected withAAV-cMG.WPP-SFFV-DIO-mScarlet (left), AAV-MG1.1-SFFV-DIO-mScarlet (middle) or AAV-MG1.2-SFFV-DIO-mScarlet (right). (B) Representative images showing the mScarlet expression patterns in the hippocampus and the thalamus of Cx3cr1^{CreER} mice injected with AAV-MG1.1-SFFV-DIO-mScarlet (left) or AAV-MG1.2-SFFV-DIO-mScarlet (right). Scale bars, 500 μm.

FIG. 5. Quantification of AAV-MG-mediated microglial transduction in vivo. (A) Cell counts of mScarlet-labeled microglia in the OFC, the striatum, and the midbrain of Cx3cr1^{CreER} mice injected with AAV-cMG.WPP-SFFV-DIO-mScarlet, AAV-MG1.1-SFFV-DIO-mScarlet, or AAV-MG1.2-SFFV-DIO-mScarlet (n=3 mice for each group; two-way ANOVA with Tukey's post-hoc test; cMG.WPP vs. MG1.1: P<0.0001 for the OFC, the striatum, and the midbrain; cMG.WPP vs. MG1.2: P<0.0001 for the OFC, the striatum, and the midbrain). The x-axis indicates the distance (μm) of brain sections from the virus injection site. (B) Distributions of the fluorescent intensity of mScarlet-labeled microglia in the striatum of Cx3cr1^{CreER} mice injected with AAV-MG1.1-SFFV-DIO-mScarlet (left) or AAV-MG1.2-SFFV-DIO-mScarlet (right) (n=3 mice for each group). The AAVs were injected with or without retro-orbital administration of doxorubicin (Doxo). (C) Representative images showing the colocalization of mScarlet and Iba⁺ immunosignals in the midbrain of Cx3cr1^{CreER} mice injected with AAV-MG1.1-SFFV-DIO-mScarlet. (D) Quantification of the percentage of the mScarlet and Iba double-positive microglia among total mScarlet-positive microglia (n=3 mice for each group). (E) Representative images showing the mScarlet expression patterns in the striatum of Cx3cr1^{CreER} mice injected with AAV5-SFFV-DIO-mScarlet (left), AAV6TM-SFFV-DIO-mScarlet (middle), or AAV9-SFFV-DIO-mScarlet (right). Scale bars, 100 μm (C), 100 μm (E).

FIG. 6. In vivo transduction of microglia by AAV-MGs does not induce microglia activation. (A) UMAPplot of 197 microglia (90 non-transduced, 11 AAV-MG1.1-transduced, and 96 AAV-MG1.2 transduced) isolated from 3 mice. The inset shows the log-normalized expression level of the mScarlet transcript. (B) Violin plots showing the expression level of homeostatic marker genes (Cx3cr1, Tmem119, P2ry12, and Csf1r) and reactive marker genes (Cd74, Tlr2, Cebpb, and Spp1). (C) A de novo, re-computed UMAP plot of the reference microglia datasets (4,500 microglia from control mice and 9,832 microglia from LPS-treated mice, sequenced using the lOx Genomics platform) and the query dataset (sequenced using the Smart-seq2 protocol). The inset summarizes the states of the Smart-seq2 sequenced microglia, as predicted by Seurat label transfer.

FIG. 7. Reference single-cell RNA sequencing datasets characterizing the single-cell transcriptomes of homeostatic and reactive microglia. (A) Quantification of the gene counts, the percentage of mitochondria RNA, and the percentage of ribosome RNA in the Smart-seq2 dataset shown in FIG. 6A. Red dots represent sequenced microglia that failed to pass the quality check and were removed from subsequent analysis. In total, 197 quality-controlled (QC)-positive single microglia (90 non-transduced, 11 AAV-MG1.1-transduced, and 96 AAV-MG1.2-transduced) were obtained, with a mean gene detection rate of 3,961 genes per cell. (B) UMAP plot of 14,332 microglia in the reference microglia datasets [4,500 from control mice and 9,832 from LPS-treated mice (0.83 mg/kg, i.p. injection), sequenced using the lOx Genomics platform]. (C) Violin plots showing the expression level of homeostatic marker genes (Cx3cr1, Tmem119, P2ry12, and Csf1r) and reactive marker genes (Cd74, Tlr2, Cebpb, and Spp1) in the reference microglia datasets. (D) Quantification of the gene expression changes in microglia between the homeostatic state and the reactive state in the reference microglia datasets. Red dots represent reactive marker genes.

FIG. 8. AAV-MGs enable in vivo two-photon imaging of microglia Ca²⁺ signal and ATP transmission. (A) Schematic showing the experimental procedure for in vivo two-photon imaging of calcium signal and ATP transmission. (B) Images showing GCaMP8s expression in microglia and heatmaps showing the GCaMP8s fluorescence signals at three hours after LPS (left) or saline (right) injection. (C) Quantification of GCaMP8s fluorescence signals at microglia somata after LPS (red) or saline (black) i.p. injection (n=14 cells for LPS group, 10 cells for saline group; two-way ANOVA, #: Tukey's post-hoc vs. 0 hour, *: Sidak's post-hoc vs. saline). (D) Images showing GRAB_{ATP1,0} expression in microglia and heatmaps showing the GRAB_{ATP1,0} fluorescence signals at three hours after LPS (left) or saline (right) injection. (E) Quantification of GRAB_{ATP1,0} fluorescence signals at microglia somata after LPS (red) or saline (black) i.p. injection (n=14 cells for LPS group, 10 cells for saline group; two-way ANOVA, #: Tukey's post-hoc vs. 0 hour, *: Sidak's post-hoc vs. saline). Error bars indicate the s.e.m. Scale bars, 100 μm (B, D).

FIG. 9. AAV-MG1.2 enables in vivo two-photon imaging of microglia extracellular ATP changes following acute laser ablation (A) Quantification of GRAB_{ATP1,0} fluorescence signals at microglia somata in the control mice (black) or in the mice that received laser ablation (red) (n=10 cells for the control group, 16 cells for the laser ablation group; two-way ANOVA). (B) Images showing GRABATPi.o expression in microglia and heatmaps showing the GRAB_{ATP1,0} fluorescence signals at 10 (left), 20 (middle), and 40 (right) mins in the imaging session. For the laser ablation group, the laser was applied at the center of field of view at the beginning (0 min) of the imaging session. Error bars indicate the s.e.m. Scale bars, 100 μm.

FIG. 10. AAV-MGs mediate efficient microglia genome editing in vivo. (A) Representative immunofluorescence images of the striatum of Cx3cr1^{CreER}:Rosa26-LSL-Cas9 mice injected with AAV-MG1.1-sgRNA-LacZ (left) or AAV-MG1.1-sgRNA-Tmem119 (right). The brain sections were immunostained against Tmem119. The upper right panel shows zoomed-in views of the boxed regions in the left panel. The bottom right panel shows the percentage of Tmem119-positive pixels in a 1.5 mm×1.5 mm region in the dorsal striatum (n=3 mice for each group; two-way ANOVA). The x-axis indicates the distance (μm) of brain sections from the virus injection site. (B) Representative immunofluorescence images of the striatum of Cx3cr1^{CreER}:Rosa26-LSL-Cas9 mice injected with AAV-MG1.2-sgRNA-LacZ (left) or AAV-MG1.2-sgRNA-Cd68 (right). The brain sections were immunostained against Cd68. The upper right panel shows zoomed-in views of the boxed regions in the left panel. The bottom right panel shows the number of Cd68-positive cells in a 1 mm×1 mm region in the dorsal striatum (n=3 mice for each group; two-way ANOVA). The x-axis indicates the distance (μm) of brain sections from the virus injection site. (C) Schematic showing the experimental procedure for in vivo two-photon imaging of microglial responses to laser ablation. (D) Quantification of microglial process extension toward the laser ablation site in Cx3cr1^{GFP} mice injected with AAV-MG1.2-CMV-SaCas9 (black) or AAV-MG1.2-CMV-SaCas9-U6-sgRNA-P2ry12 (red) (n=4 fields from 3 mice injected with AAV-MG1.2-CMV-SaCas9, 6 fields from 3 mice injected with AAV-MG1.2-CMV-SaCas9-U6-sgRNA-P2ry12; two-way ANOVA). (E) Representative images showing the recruitment of microglial processes to the two-photon laser ablation site in Cx3cr1^{GFP} mice injected with AAV-MG1.2-CMV-SaCas9 (left) or AAV-MG1.2-CMV-SaCas9-U6-sgRNA-P2ry12 (right). Error bars indicate the s.e.m. Scale bars, 200 μm (A left, B left), 100 μm (A right, B right), 20 μm (E).

FIG. 11. In vivo microglia-specific gene knockout mediated by AAV-MGs. (A) Representative immunofluorescence images of the striatum of Cx3cr1^{CreER}:Rosa26-LSL-Cas9 mice injected with AAV-MG1.1-sgRNA-LacZ (top) or AAV-MG1.1-sgRNA-Tmem119 (bottom). The brain sections were immunostained against Tmem119. (B) Representative immunofluorescence images of the striatum of Cx3cr1^{CreER}:Rosa26-LSL-Cas9 mice injected with AAV-MG1.2-sgRNA-LacZ (top) or AAV-MG1.2-sgRNA-Cd68 (bottom). The brain sections were immunostained against Cd68. (C) Representative images of the Si cortex of Cx3cr1^{GFP} mice injected with AAV-MG1.2-CMV-SaCas9 (top) or AAV-MG1.2-CMV-SaCas9-U6-sgRNA-P2ry12 (bottom). The brain sections were immunostained against P2ry12. (D) Quantification of the percentage of P2ry12-positive pixels in a 1 mm×1 mm region in the S1 cortex of Cx3cr1^{GFP} mice injected with AAV-MG1.2-CMV-SaCas9 (black) or AAV-MG1.2-CMV-SaCas9-sgRNA-P2ry12 (red) (n=3 mice for P2ry12-knockout group; 2 mice for control group; two-way ANOVA). Error bars indicate the s.e.m. Scale bars, 500 μm (A and B), 100 μm (C).

FIG. 12. Representative images showing the mScarlet expression patterns in the striatum of Cx3cr1^{CreER} mice injected with (A) AAV-MG.TAF-SFFV-DIO-mScarlet, (B) AAV-MG.APA-SFFV-DIO-mScarlet, (C) AAV-MG. PTS-SFFV-DIO-mScarlet, (D) AAV-MG.LMV-SFFV-DIO-mScarlet, (E) AAV-MG.WTD-SFFV-DIO-mScarlet, or (F) AAV-MG.VLS-SFFV-DIO-mScarlet. Scale bars, 500 μm.

### DETAILED DESCRIPTION OF THE INVENTION

Before the present methods and compositions are described, it is to be understood that this invention is not limited to a particular method or composition described and as such may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by those skilled in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It is noted that as used herein and in the appended claims, the singular forms “a,” “an,” and “the” include plural referents unless the context clearly dictates otherwise. Thus, for example, reference to “a recombinant AAV virion” includes a plurality of such virions and reference to “microglia” includes reference to one or more microglia cells and equivalents thereof known to those skilled in the art, and so forth.

### Definitions

Unless otherwise defined, all scientific and technical terms used herein have the same meaning as commonly understood by those skilled in the art to which this technology belongs.

Adeno-associated virus (AAV) is a member of the Parvoviridae, belonging to the Dependovirus genus. AAV is a nonpathogenic parvovirus composed of a single-stranded DNA genome of approximately 4.7 kb within a non-enveloped, icosahedral capsid. The genome contains three open reading frames (ORF) flanked by inverted terminal repeats (ITR) that function as the viral origin of replication and packaging signal. The rep ORF encodes four nonstructural proteins that play roles in viral replication, transcriptional regulation, site-specific integration, and virion assembly. The cap ORF encodes three structural proteins (VPs 1-3) that assemble to form a 60-mer viral capsid. Finally, an ORF present as an alternate reading frame within the cap gene produces the assembly-activating protein (AAP), a viral protein that localizes AAV capsid proteins to the nucleolus and functions in the capsid assembly process. Based on crystal structures of AAV, the VP amino acids involved in forming the icosahedral fivefold, threefold, and twofold symmetry interfaces have been visualized. The surface loops at the threefold axis of symmetry are thought to be involved in host cell receptor binding and have been the target of mutagenesis studies.

There are several naturally occurring (“wild-type”) serotypes and over 100 known variants of AAV, each of which differs in amino acid sequence, particularly within the hypervariable regions of the capsid proteins, and thus in their gene delivery properties. No AAV has been associated with any human disease, making recombinant AAV attractive for clinical applications.

Otherwise indicated, the term “adeno-associated virus” or “AAV” refers to all subtypes or serotypes and both replication-competent and recombinant forms. The term “AAV” includes, without limitation, AAV type 1 (AAV-1 orAAV1), AAV type 2 (AAV-2 or AAV2), AAV type 3A (AAV-3A or AAV3A), AAV type 3B (AAV-3B or AAV3B), AAV type 4 (AAV-4 or AAV4), AAV type 5 (AAV-5 or AAV5), AAV type 6 (AAV-6 or AAV6), AAV type 7 (AAV-7 or AAV7), AAV type 8 (AAV-8 or AAV8), AAV type 9 (AAV-9 or AAV9), AAV type 10 (AAV-10 or AAV 10 or AAVrh10), avian AAV, bovine AAV, canine AAV, caprine AAV, equine AAV, primate AAV, non-primate AAV, and ovine AAV. “Primate AAV” refers to AAV that infect primates, “non-primate AAV” refers to AAV that infect non-primate mammals, “bovine AAV” refers to AAV that infect bovine mammals and the like.

The genomic sequences of various serotypes of AAV, as well as the sequences of the native terminal repeats (TRs), Rep proteins, and capsid subunits are known in the art. Such sequences may be found in the literature or in public databases such as GenBank. See, e.g., GenBank Accession Numbers NC_002077.1 (AAV1), AF063497.1 (AAV1), NC_001401.2 (AAV2), AF043303.1 (AAV2), J01901.1 (AAV2), U48704.1 (AAV3A), NC_001729.1 (AAV3A), AF028705.1 (AAV3B), NC 0.001829.1 (AAV4), U89790.1 (AAV4), NC_006152.1 (AA5), AF085716.1 (AAV-5), AF028704.1 (AAV6), NC 006260.1 (AAV7), AF513851.1 (AAV7), AF513852.1 (AAV8) NC 006261.1 (AAV-8), AY530579.1 (AAV9), AAT46337 (AAV10) and AAO88208 (AAVrh10); the disclosures of which are incorporated by reference herein for teaching AAV polynucleotide and amino acid sequences.

The term “recombinant adeno-associated virus capsid protein” or “rAAV capsid protein” as used herein refers to an AAV capsid protein comprising a seven-amino-acid peptide insertion in a GH-loop of the VP1-VP3 capsid protein as compared to a wide-type VP1-VP3 capsid protein thereof.

The term “recombinant adeno-associated virus virion(s)”, “rAAV virion(s)”, “rAAV vector(s)” or “rAAV particles” as used herein refers to a viral particle comprising a recombinant/variant capsid protein.

If an AAV vector/virion comprises a heterologous polynucleotide sequence, the heterologous polynucleotide sequence refers to a polypolynucleotide sequence other than a wild-type AAV genome, e.g., a transgene to be delivered to a target cell, an RNAi agent or CRISPR agent to be delivered to a target cell, and the like. In general, the heterologous polynucleotide sequence is flanked by at least one, and generally by two, AAV inverted terminal repeat sequences (ITRs).

The term “heterologous” as used herein means derived from a genotypically distinct entity from that of the rest of the entity to which it is being compared. For example, a polynucleotide introduced by genetic engineering techniques into a plasmid or vector derived from a different species is a heterologous polynucleotide. Thus, for example, an rAAV that includes a heterologous nucleic acid sequence encoding a heterologous gene product is an rAAV that includes a polynucleotide not normally included in a naturally-occurring, wild-type AAV, and the encoded heterologous gene product is a gene product not normally encoded by a naturally-occurring, wild type AAV.

The term “packaging” as used herein refers to a series of intracellular events that result in the assembly and encapsidation of an AAV particle. AAV “rep” and “cap” genes refer to polypolynucleotide sequences encoding replication and encapsidation proteins of adeno-associated virus. AAV rep and cap are referred to herein as AAV “packaging genes”.

The term “polynucleotide” as used herein refers to a polymeric form of nucleotides of any length, including deoxyribonucleotides or ribonucleotides, or analogs thereof. A polynucleotide may comprise modified nucleotides, such as methylated nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment herein that comprises a polynucleotide encompasses both the double-stranded form and each of two complementary single-stranded forms known or predicted to make up the double-stranded form.

The term “gene” as used herein refers to a polynucleotide that performs a function of some kind in the cell. For example, a gene can contain an open reading frame that is capable of encoding a gene product. One example of a gene product is a protein, which is transcribed and translated from the gene. Another example of a gene product is an RNA, e.g. a functional RNA product, e.g., an aptamer, an interfering RNA, a ribosomal RNA (rRNA), a transfer RNA (tRNA), a non-coding RNA (ncRNA), a guide RNA for nucleases and the like, which is transcribed but not translated.

With regards to “CRISPR/Cas9 agents”, the term “CRISPR” encompasses Clustered regularly interspaced short palindromic repeats/CRISPR-associated (Cas) systems that evolved to provide bacteria and archaea with adaptive immunity against viruses and plasm ids by using CRISPR RNAs (crRNAs) to guide the silencing of invading nucleic acids. The Cas9 protein (or functional equivalent and/or variant thereof, i.e., Cas9-like protein) naturally contains DNA endonuclease activity that depends on association of the protein with two naturally occurring or synthetic RNA molecules called crRNA and tracrRNA (also called guide RNAs). In some cases, the two molecules are covalently linked to form a single molecule (also called a single guide RNA (“sgRNA”)). Thus, the Cas9 or Cas9-like protein associates with a DNA-targeting RNA (which term encompasses both the two-molecule guide RNA configuration and the single-molecule guide RNA configuration), which activates the Cas9 or Cas9-like protein and guides the protein to a target nucleic acid sequence. If the Cas9 or Cas9-like protein retains its natural enzymatic function, it will cleave target DNA to create a double-strand break, which can lead to genome alteration (i.e., editing: deletion, insertion (when a donor polynucleotide is present), replacement, etc.), thereby altering gene expression.

The term “CRISPR agent” as used herein encompasses any agent (or nucleic acid encoding such an agent), comprising naturally occurring and/or synthetic sequences, that can be used in the Cas9-based system (e.g., a Cas9 or Cas9-like protein; any component of a DNA-targeting RNA, e.g., a crRNA-like RNA, a tracrRN A-like RNA, a single guide RNA, etc.; a donor polynucleotide; and the like).

The terms “treatment”, “treating” and the like as used herein, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease.

The terms “individual”, “host”, “subject”, and “patient” are used interchangeably herein, and refer to a mammal, including, but not limited to, humans; non-human primates, including simians; mammalian sport animals (e.g., horses); mammalian farm animals (e.g., sheep, goats, etc.); mammalian pets (dogs, cats, etc.); and rodents (e.g., mice, rats, etc.).

The term “microglia” as used herein means the cells of mesodermal/mesenchymal origin that migrate into the CNS to become resident macrophages within the unique brain microenvironment. Microglia are highly dynamic cells that interact with neurons and non-neuronal cells. Microglia patrol the brain parenchyma via continuous process extension and retraction and are also capable of transitioning from a ramified to an ameboid morphology, a feature that is consistent with cell activation. Microglia express a wide array of receptors and thus respond to pleiotropic stimuli ranging from neurotransmitters to cytokines and plasma proteins. They play a crucial role in the healthy brain as regulators of synaptic functions and phagocytosis of newborn neurons, with important implications in synaptic plasticity and adult neurogenesis. In disease, they play a crucial role in neurological and neuroinflammatory conditions. Their interactions with T cells are a major component of the development of brain autoimmunity, while their pathogenic interactions with neurons via induction of ROS and iNOS play a crucial role in neurological disorders. Emerging genetic tools and animal models have shed new light on the origin of microglia, their link to peripheral monocytes, and their contribution to disease pathogenesis. As microglia might exert beneficial and pathogenic functions in the CNS, understanding their contribution in disease-specific contexts will be necessary for the identification of novel microglia-targeted therapies for CNS diseases.

The term “directed evolution” as used herein refers to a capsid engineering methodology, in vitro and/or in vivo, which emulates natural evolution through iterative rounds of genetic diversification and selection processes, thereby accumulating beneficial mutations that progressively improve the function of a biomolecule. Directed evolution often involves an in vivo method referred to as “biopanning” for selection of AAV variants from a library which variants possess a more efficient level of infectivity of a cell or tissue type of interest.

With regards to cell modification, the term “genetically modified” or “transformed” or “transfected” or “transduced” by exogenous DNA (e.g. via a recombinant virus) refers to when such DNA has been introduced inside the cell. The presence of the exogenous DNA results in permanent or transient genetic change. The transforming DNA may or may not be integrated (covalently linked) into the genome of the cell.

Without being bound by theory, the present disclosure is based in part on the surprising finding of several new AAV capsids, which mediate efficient gene delivery to microglia, with screening processes.

Recombinant Adeno-Associated Virus (rAAV) Vector

Adeno-associated viruses (AAVs) are a family of parvoviruses with a 4.7 kb single-stranded DNA genome contained inside a non-enveloped capsid. The viral genome of a naturally occurring AAV has 2 inverted terminal repeats (ITR)—which function as the viral origin of replication and packaging signal—flanking 2 primary open reading frames (ORF): rep (encoding proteins that function in viral replication, transcriptional regulation, site-specific integration, and virion assembly) and cap. The cap ORF codes for 3 structural proteins that assemble to form a 60-mer viral capsid. Many naturally occurring AAV variants and serotypes have been isolated, and none have been associated with human disease.

Recombinant versions of AAV can be used as gene delivery vectors, where a marker or therapeutic gene of interest is inserted between the ITRs in place of rep and cap. These vectors have been shown to transduce both dividing and non-dividing cells in vitro and in vivo and can result in stable transgene expression for years in post-mitotic tissue.

Recombinant AAV (rAAV) has yielded promising results in an increasing number of clinical trials. However, there are impediments to gene delivery that may limit AAV's utility, such as anti-capsid immune responses, low transduction of certain tissues, an inability for targeted delivery to specific cell types and a relatively low carrying capacity. In many situations, there is insufficient mechanistic knowledge to effectively empower rational design with the capacity to improve AAV. As an alternative, directed evolution has emerged as a strategy to create novel AAV variants that meet specific biomedical needs. Directed evolution strategies harness genetic diversification and selection processes to enable the accumulation of beneficial mutations that progressively improve the function of a biomolecule. In this process, wild-type AAV cap genes are diversified by several approaches to create large genetic libraries that are packaged to generate libraries of viral particles, and selective pressure is then applied to isolate novel variants that can overcome gene delivery barriers. Importantly, the mechanistic basis underlying a gene delivery problem does not need to be known for directed evolution of function, which can thus accelerate the development of enhanced vectors.

Three AAV capsid proteins (i.e., VP1, VP2 and VP3) are produced in an overlapping fashion from the cap ORF by using alternative mRNA splicing of the transcript and alternative translational start codon usage. A common stop codon is employed for all three capsid proteins. Though only VP1 is illustrated in the examples and drawings, it should be understood that each of VP1, VP2 and VP3 comprises the amino acid sequence of 11 contiguous amino acids X₁X₂X₃X₄X₅X₆X₇X₈X₉X₁₀Q provided by the present disclosure.

In some embodiments, the amino acid sequence of 11 contiguous amino acids X₁X₂X₃X₄X₅X₆X₇X₈X₉X₁₀Q provided by the present disclosure is inserted in a GH-loop of VP1, VP2 and/or VP3 capsid proteins, as compared to a wide-type VP1-VP3 capsid proteins thereof.

Regarding VP1 capsid protein, the above amino acid sequence is inserted between amino acids 588 and 589 of the wide-type VP1 of AAV9 or the corresponding position in the capsid protein of another AAV serotype than AAV9. In some embodiments, the other AAV serotypes may comprise AAV2, AAV1, AAV3A, AAV3B, AAV4, AAV5, AAV6, AAV7, AAV8, AAV10 and the like. In some embodiments, the peptide insertion may be located between amino acids 587 and 588 of AAV2. In some embodiments, the peptide insertion may be located between amino acids 591 and 592 of AAV1. In some embodiments, the peptide insertion may be located between amino acids 588 and 589 of AAV3A. In some embodiments, the peptide insertion may be located between amino acids 588 and 589 of AAV3B. In some embodiments, the peptide insertion may be located between amino acids 584 and 585 of AAV4. In some embodiments, the peptide insertion may be located between amino acids 575 and 576 of AAV5. In some embodiments, the peptide insertion may be located between amino acids 591 and 592 of AAV6. In some embodiments, the peptide insertion may be located between amino acids 589 and 590 of AAV7. In some embodiments, the peptide insertion may be located between amino acids 591 and 592 of AAV8. In some embodiments, the peptide insertion may be located between amino acids 588 and 589 of AAV10.

In some embodiments, the variant VP1 capsid protein, from amino acids 587 to 597 of AAV9 or a corresponding position in the capsid protein of another AAV serotype than AAV9, comprises the amino acid sequence of 11 contiguous amino acids X₁X₂X₃X₄X₅X₆X₇X₈X₉X₁₀Q provided by the present disclosure.

In some embodiments, the wide-type VP1 capsid protein of AAV9 comprises an amino acid sequence as shown by SEQ ID NO: 6.

In some embodiments, the variant VP1 capsid protein of the rAAV comprises an amino acid sequence as shown by SEQ ID NO: 7. In some embodiments, the variant VP1 capsid protein of the rAAV comprises an amino acid sequence as shown by SEQ ID NO: 8. In some embodiments, the variant VP1 capsid protein of the rAAV comprises an amino acid sequence as shown by SEQ ID NO: 9. In some embodiments, the variant VP1 capsid protein of the rAAV comprises an amino acid sequence as shown by SEQ ID NO: 10. In some embodiments, the variant VP1 capsid protein of the rAAV comprises an amino acid sequence as shown by SEQ ID NO: 11. In some embodiments, the variant VP1 capsid protein of the rAAV comprises an amino acid sequence as shown by SEQ ID NO: 53. In some embodiments, the variant VP1 capsid protein of the rAAV comprises an amino acid sequence as shown by SEQ ID NO: 56. In some embodiments, the variant VP1 capsid protein of the rAAV comprises an amino acid sequence as shown by SEQ ID NO: 59. In some embodiments, the variant VP1 capsid protein of the rAAV comprises an amino acid sequence as shown by SEQ ID NO: 62. In some embodiments, the variant VP1 capsid protein of the rAAV comprises an amino acid sequence as shown by SEQ ID NO: 65. In some embodiments, the variant VP1 capsid protein of the rAAV comprises an amino acid sequence as shown by SEQ ID NO: 68.

Further, according to a specific embodiment, the variant VP1 capsid protein comprising an amino acid sequence as shown by SEQ ID NO.: 10, 11 59, 62, 65 or 68 has significantly higher transduction ability for microglia in vivo, as compared to the wide type VP1 capsid or variant VP1 capsid protein comprising an amino acid sequence as shown by SEQ ID NO.: 7. Further, according to a specific embodiment, the variant VP1 capsid protein comprising an amino acid sequence as shown by SEQ ID NO.: 9, 53 or 56 has significantly higher transduction ability for microglia in vivo, as compared to the wide type VP1 capsid or variant VP1 capsid protein comprising an amino acid sequence as shown by SEQ ID NO.: 8.

Regarding VP2 capsid protein, the above amino acid sequence of 11 contiguous amino acids X₁X₂X₃X₄X₅X₆X₇X₈X₉X₁₀Q is inserted between amino acids 451 and 452 of the wide-type VP2 of AAV9 or the corresponding position in the VP2 capsid protein of another AAV serotype than AAV9. In some embodiments, the variant VP2 capsid protein, from amino acids 450 to 460 of AAV9 or a corresponding position in the VP2 capsid protein of another AAV serotype than AAV9, comprises the amino acid sequence of 11 contiguous amino acids X₁X₂X₃X₄X₅X₆X₇X₈X₉X₁₀Q provided by the present disclosure. The definitions relating to X₁ to X₁₀ are the same as the above.

In some embodiments, the wide-type VP2 capsid protein of AAV9 comprises an amino acid sequence as shown by SEQ ID NO: 41.

In some embodiments, the variant VP2 capsid protein of the rAAV comprises an amino acid sequence as shown by SEQ ID NO: 43. In some embodiments, the variant VP2 capsid protein of the rAAV comprises an amino acid sequence as shown by SEQ ID NO: 45. In some embodiments, the variant VP2 capsid protein of the rAAV comprises an amino acid sequence as shown by SEQ ID NO: 47. In some embodiments, the variant VP2 capsid protein of the rAAV comprises an amino acid sequence as shown by SEQ ID NO: 49. In some embodiments, the variant VP2 capsid protein of the rAAV comprises an amino acid sequence as shown by SEQ ID NO: 51. In some embodiments, the variant VP2 capsid protein of the rAAV comprises an amino acid sequence as shown by SEQ ID NO: 54. In some embodiments, the variant VP2 capsid protein of the rAAV comprises an amino acid sequence as shown by SEQ ID NO: 57. In some embodiments, the variant VP2 capsid protein of the rAAV comprises an amino acid sequence as shown by SEQ ID NO: 60. In some embodiments, the variant VP2 capsid protein of the rAAV comprises an amino acid sequence as shown by SEQ ID NO: 63. In some embodiments, the variant VP2 capsid protein of the rAAV comprises an amino acid sequence as shown by SEQ ID NO: 66. In some embodiments, the variant VP2 capsid protein of the rAAV comprises an amino acid sequence as shown by SEQ ID NO: 69.

Further, according to a specific embodiment, the variant VP2 capsid protein comprising an amino acid sequence as shown by SEQ ID NO.: 49, 51, 60, 63, 66 or 69 has significantly higher transduction ability for microglia in vivo, as compared to the wide type VP2 capsid protein or variant VP2 capsid protein comprising an amino acid sequence as shown by SEQ ID NO.: 43. Further, according to a specific embodiment, the variant VP2 capsid protein comprising an amino acid sequence as shown by SEQ ID NO.: 47, 54 or 57 has significantly higher transduction ability for microglia in vivo, as compared to the wide type VP2 capsid protein or variant VP2 capsid protein comprising an amino acid sequence as shown by SEQ ID NO.: 45.

Regarding VP3 capsid protein, the above amino acid sequence of 11 contiguous amino acids X₁X₂X₃X₄X₅X₆X₇X₈X₉X₁₀Q is inserted between amino acids 386 and 387 of the wide-type VP3 of AAV9 or the corresponding position in the VP3 capsid protein of another AAV serotype than AAV9. In some embodiments, the variant VP3 capsid protein, from amino acids 385 to 395 of AAV9 or a corresponding position in the VP3 capsid protein of another AAV serotype than AAV9, comprises the amino acid sequence of 11 contiguous amino acids X₁X₂X₃X₄X₅X₆X₇X₈X₉X₁₀Q provided by the present disclosure.

In some embodiments, the wide-type VP3 capsid protein of AAV9 comprises an amino acid sequence as shown by SEQ ID NO: 42.

In some embodiments, the variant VP3 capsid protein of the rAAV comprises an amino acid sequence as shown by SEQ ID NO: 44. In some embodiments, the variant VP3 capsid protein of the rAAV comprises an amino acid sequence as shown by SEQ ID NO: 46. In some embodiments, the variant VP3 capsid protein of the rAAV comprises an amino acid sequence as shown by SEQ ID NO: 48. In some embodiments, the variant VP3 capsid protein of the rAAV comprises an amino acid sequence as shown by SEQ ID NO: 50. In some embodiments, the variant VP3 capsid protein of the rAAV comprises an amino acid sequence as shown by SEQ ID NO: 52. In some embodiments, the variant VP3 capsid protein of the rAAV comprises an amino acid sequence as shown by SEQ ID NO: 55. In some embodiments, the variant VP3 capsid protein of the rAAV comprises an amino acid sequence as shown by SEQ ID NO: 58. In some embodiments, the variant VP3 capsid protein of the rAAV comprises an amino acid sequence as shown by SEQ ID NO: 61. In some embodiments, the variant VP3 capsid protein of the rAAV comprises an amino acid sequence as shown by SEQ ID NO: 64. In some embodiments, the variant VP3 capsid protein of the rAAV comprises an amino acid sequence as shown by SEQ ID NO: 67. In some embodiments, the variant VP3 capsid protein of the rAAV comprises an amino acid sequence as shown by SEQ ID NO: 70.

Further, according to a specific embodiment, the variant VP3 capsid protein comprising an amino acid sequence as shown by SEQ ID NO.: 50, 52, 61, 64, 67 or 70 has significantly higher transduction ability for microglia in vivo, as compared to the wide type VP3 capsid protein or variant VP3 capsid protein comprising an amino acid sequence as shown by SEQ ID NO.: 44. Further, according to a specific embodiment, the variant VP3 capsid protein comprising an amino acid sequence as shown by SEQ ID NO.: 48, 55 or 58 has significantly higher transduction ability for microglia in vivo, as compared to the wide typeVP3 capsid protein or variant VP3 capsid protein comprising an amino acid sequence as shown by SEQ ID NO.: 46.

In some embodiments, the rAAV vectors/virions containing the above rAAV capsid proteins, have significantly transduction efficiency of microglia, both in vitro and in vivo, as compared to parental AAV virions which do not have such the seven-amino-acid peptide insertion. In some specific embodiments, the microglia may be cultured microglia, or be microglia in brains or spinal cord.

Further, according to some embodiments, the variant VP1-VP3 capsid proteins comprising an amino acid sequence as shown by SEQ ID NO.: 2, 3, 71, 72, 73 or 74 has significantly higher transduction ability for microglia in vivo, as compared to the wide type VP1-VP3 capsid proteins or variant VP1-VP3 capsid proteins comprising an amino acid sequence as shown by SEQ ID NO.: 1.

Further, according to some embodiments, the variant VP1-VP3 capsid proteins comprising an amino acid sequence as shown by SEQ ID NO.: 5, 75 or 76 has significantly higher transduction ability for microglia in vivo, as compared to the wide type VP1-VP3 capsid proteins or variant VP1-VP3 capsid proteins comprising an amino acid sequence as shown by SEQ ID NO.: 4.

Typically, the variants disclosed herein were generated through use of an AAV library and/or libraries. Such an AAV library or libraries is/are generated by mutating the cap gene, the gene which encodes the structural proteins (e.g. VP1-VP3) of the AAV capsid, by a range of directed evolution techniques known by and readily available to the skilled artisan in the field of viral genome engineering, e.g. Cre recombination-based AAV targeted evolution (CREATE).

The CREATE process as used herein enables the development of AAV capsids that more efficiently transduce defined Cre-expressing cell populations in vivo or in vitro. To implement the CREATE strategy, a Cre recombinase targeting sequence is added in the genome of the AAV variants in the library. Without limitation, the CREATE process comprises a step of delivering the AAV library into cells or transgenic mice including target cells that selectively express Cre recombinase. For those rAAVs that successfully transduce target cells, their genome will be modified by Cre recombinase. For those rAAVs that transduce non-target cells, their genomes will not be modified. In such way, specific primers can be used to selectively recover the genomes of AAV variants that are capable of transducing target cells.

Once the AAV library or libraries is/are generated, viruses are then packaged, such that each AAV particle is comprised of a mutant VP1, VP2 and/or VP3 capsid proteins. Variants of the library are then subjected to in vitro and/or in vivo selective pressure techniques known by and readily available to the skilled artisan in the field of AAV. For example, without limitation, AAV variants can be selected using i) affinity columns in which elution of different fractions yields variants with altered binding properties; ii) primary cells—isolated from tissue samples or immortal cells lines that mimic the behavior of cells in the human body, which yield AAV variants with increased efficiency and/or tissue specificity; iii) animal models—which mimic a clinical gene therapy environment—which yield AAV variants that have successfully infected target tissue; iv) human xenograft models which yield AAV variants that have infected grafted human cells; and/or a combination of selection techniques thereof.

Once viruses are selected, they may be recovered by known techniques such as, without limitation, adenovirus-mediated replication, PCR amplification, Next Generation sequencing and cloning, and the like. Virus clones are then enriched through repeated rounds of the selection techniques and AAV DNA is isolated to recover selected variant cap genes of interest. Such selected variants can be subjected to further modification or mutation and as such serve as a new starting point for further selection steps to iteratively increase AAV viral fitness. However, in certain instances, successful capsids have been generated without additional mutation.

The AAV variants disclosed herein were generated at least in part through the use of in vitro or in vivo directed evolution methodology, such as the techniques described above, involving the use of screening in cultured primary mouse microglia cells or in vivo microglia following injecting into the striatum and/or midbrain of the mice. As such, the AAV variant capsids disclosed herein comprise a seven-amino-acid peptide insertion in a GH-loop of VP1, VP2 and/or VP3 that confer more efficient transduction than a corresponding parental AAV capsid protein or control. As used herein, a “corresponding parental AAV capsid protein” refers to an AAV capsid protein of the same wild-type or variant AAV serotype as the subject variant AAV capsid protein but that does not comprise the peptide insertion of the subject variant AAV capsid protein.

In some embodiments, the subject variant AAV capsid protein comprises a heterologous peptide of 7 amino acids inserted by covalent linkage into an AAV capsid protein G-H loop, or loop IV, relative to a corresponding parental AAV capsid protein. By the “G-H loop,” or loop IV, of the AAV capsid protein it is meant the solvent-accessible portion referred to in the art as the GH loop, or loop IV, of AAV capsid protein.

In certain embodiments, the insertion site is a single insertion site between two adjacent amino acids located between amino acids 570 and 614 of VP1, amino acids 451 and 452 of VP2, and/or amino acids 386 and 387 of VP3, of any wild-type AAV serotype or AAV variant.

For example, the insertion site is between two adjacent amino acids located in amino acids 570-610, amino acids 580-600, amino acids 570-575, amino acids 575-580, amino acids 580-585, amino acids 585-590, amino acids 590-600, or amino acids 600-614, of VP1 of any AAV serotype or variant. In a preferred embodiment, the insertion site is between amino acids 580 and 581, amino acids 581 and 582, amino acids 583 and 584, amino acids 584 and 585, amino acids 585 and 586, amino acids 586 and 587, amino acids 587 and 588, amino acids 588 and 589, or amino acids 589 and 590. The insertion site can be between amino acids 575 and 576, amino acids 576 and 577, amino acids 577 and 578, amino acids 578 and 579, or amino acids 579 and 580. The insertion site can be between amino acids 590 and 591, amino acids 591 and 592, amino acids 592 and 593, amino acids 593 and 594, amino acids 594 and 595, amino acids 595 and 596, amino acids 596 and 597, amino acids 597 and 598, amino acids 598 and 599, or amino acids 599 and 600. In a preferred embodiment, the insertion site is between amino acids 587 and 588 of AAV2, between amino acids 591 and 592 of AAV1, between amino acids 588 and 589 of AAV3A, between amino acids 588 and 589 of AAV3B, between amino acids 584 and 585 of AAV4, between amino acids 575 and 576 of AAV5, between amino acids 591 and 592 of AAV6, between amino acids 589 and 590 of AAV7, between amino acids 591 and 592 of AAV8, between amino acids 588 and 589 of AAV9, or between amino acids 588 and 589 of AAV10. Those skilled in the art would know, based on a comparison of the amino acid sequences of capsid proteins of various AAV serotypes, where an insertion site “corresponding to amino acids of AAV9” would be in a capsid protein of any given AAV serotype.

The peptide insertions disclosed herein have not been previously described and/or inserted into an AAV capsid. Without wishing to be bound by theory, the presence of any of the disclosed peptide insertions may act to increase the transduction of the AAV into microglia, both in vitro or in vivo.

In certain embodiments, for the amino acid sequence X₁X₂X₃X₄X₅X₆X₇X₈X₉X₁₀Q, X₁ is selected from Ala or Leu; X₂ is selected from Gln, Met, Thr, Val or Pro; X₃ is selected from Trp, Thr, Glu, Pro, Leu, Ala or Gln; X₄ is selected from Pro, Thr, Met, Ser, Arg or Ala; X₅ is selected from Pro, Ser, Val, Asp or Phe; X₆ is selected from Lys or Pro; X₇ is selected from Thr or Arg; X₈ is selected from Thr, Glu or Pro; X₉ is selected from Ser, Pro or Ala; and X₁₀ is selected from Ala or Asp.

In certain embodiments, the variant VP1, VP2 and/or VP3 capsid protein comprises an amino acid sequence AQWPPKTTSAQ (SEQ ID NO.: 1), LMTPPKTTSAQ (SEQ ID NO.: 2), ATEPPKTTSAQ (SEQ ID NO.: 3), AQQRPPREPAQ (SEQ ID NO.: 4), AQQRPPRPADQ (SEQ ID NO.: 5), AQPTSKTTSAQ (SEQ ID NO.: 71), AQLMVKTTSAQ (SEQ ID NO.: 72), AQWTDKTTSAQ (SEQ ID NO.: 73), AVLSPKTTSAQ (SEQ ID NO.: 74), AQTAFPREPAQ (SEQ ID NO.: 75) or APARPPREPAQ (SEQ ID NO.: 76).

In some embodiments, the rAAV virion can be produced by co-transfecting a plasmid expressing the variant capsid proteins of the present disclosure, an adenoviral helper plasmid, and optionally a transgene plasmid expressing the heterologous polynucleotide sequence into 293/293T cells.

In some embodiments, the rAAV virion comprises a heterologous polynucleotide sequence which encodes a gene product. In some embodiments, the gene product is an interfering RNA. In some embodiments, the gene product is a long or short non-coding RNA. In some embodiments, the gene product is an antisense RNA. In some embodiments, the gene product is a guide RNA. In some embodiments, the gene product is an aptamer. In some embodiments, the gene product is a polypeptide. In some embodiments, the gene product is a secreted antibody. In some embodiments, the gene product is a single chain antibody. In some embodiments, the gene product is a VHH domain. In some embodiments, the gene product is a soluble receptor. In some embodiments, the gene product is an affibody. In some embodiments, the gene product is a chaperone. In some embodiments, the gene product is a site-specific nuclease that provide for site-specific knock-down of gene function.

In some embodiments, the rAAV virion of the present disclosure has improved transduction rate and stronger expression of the heterologous polynucleotide sequence, as compared to the parental AAV virion and other wide-type AAV serotypes.

In some embodiments, the rAAV virion of the present disclosure is transduced into cultured microglia without inducing the proinflammatory pathways in the microglia.

In a certain embodiment, the rAAV9 virion, which comprises a variant VP1, VP2 and/or VP3 capsid protein including an amino acid sequence of AQWPPKTTSAQ (SEQ ID NO.: 1) or AQQRPPREPAQ (SEQ ID NO.: 4) has improved transduction rate and stronger expression of the heterologous polynucleotide sequence in cultured microglia, as compared to the parental AAV9 virion and other wide-type AAV serotypes.

In a certain embodiment, the rAAV9 virion, which comprises a variant VP1, VP2 and/or VP3 capsid protein including an amino acid sequence of LMTPPKTTSAQ (SEQ ID NO.: 2), ATEPPKTTSAQ (SEQ ID NO.: 3), AQPTSKTTSAQ (SEQ ID NO.: 71), AQLMVKTTSAQ (SEQ ID NO.: 72), AQWTDKTTSAQ (SEQ ID NO.: 73) or AVLSPKTTSAQ (SEQ ID NO.: 74) has even further improved transduction rate and stronger expression of the heterologous polynucleotide sequence in microglia in vivo, as compared to rAAV9 virion including a peptide insertion of AQWPPKTTSAQ (SEQ ID NO.: 1), as well as the parental AAV9 virion and other wide-type AAV serotypes.

In a certain embodiment, the rAAV9 virion, which comprises a variant VP1, VP2 and/or VP3 capsid protein including an amino acid sequence of AQQRPPRPADQ (SEQ ID NO.: 5), AQTAFPREPAQ (SEQ ID NO.: 75) or APARPPREPAQ (SEQ ID NO.: 76) has even further improved transduction rate and stronger expression of the heterologous polynucleotide sequence in microglia in vivo, as compared to rAAV9 virion including an amino acid sequence of AQQRPPREPAQ (SEQ ID NO.: 4), as well as the parental AAV9 virion and other wide-type AAV serotypes.

In some embodiments, the rAAV-mediated transgene expression in microglia can be further increased by pharmacological approaches. In a certain embodiments, the topoisomerase and proteasome inhibitor is used for further increasing the expression level of the heterologous nucleotide sequence, which is transduced by using the rAAV virion of the present disclosure. In a preferred embodiment, a topoisomerase inhibitor, e.g. doxorubicin, is used for increasing the expression level of the heterologous nucleotide sequence, which is transduced by using the rAAV virion of the present disclosure.

In some embodiments, the transduction mediated by the rAAV virion of the present disclosure does not induce microglia activation, both in vitro and in vivo.

In some embodiments, the rAAV virion of the present disclosure carries a heterologous nucleotide sequence encoding a CRISPR agent for genome editing, e.g. the knock out of a gene, both in vitro and in vivo. In some embodiments, the rAAV virion of the present disclosure carries a heterologous nucleotide sequence encoding a guide RNA.

The following examples are set forth to provide the ordinarily skilled artisan with a complete disclosure and description for guidance as to how to make and use the variant AAV capsids disclosed herein, and are not intended to limit the scope of the invention disclosed herein. In addition, the following examples are not intended to represent that the experiments below are all or the only experiments.

### METHODS

Mice. Animal care and use followed the approval of the Animal Care and Use Committee of the National Institute of Biological Sciences, Beijing in accordance with the Regulations for the Administration of Affairs Concerning Experimental Animals of China. Cx3cr1^{CreER} mice (021160, B6.129P2(Cg)-Cx3cr1tm2.1(cre/ERT2)Litt/WganJ), Cx3cr1GFP mice (005582, B6.129P2(Cg)-Cx3cr1tm1Litt/J), and Rosa26-LSL-Cas9 mice (024857, B6; 129-Gt(ROSA)26Sortm](CAG-cas9*-EGFP)Fezh/J) mice were obtained from Jackson Laboratory. Adult mice of either sex were used for in vivo virus injection. The postnatal day 1 (P1) and adult C57BL/6N wildtype mice were obtained from Beijing Vital River Laboratory Animal Technology. Mice were maintained with a 12/12 hour photoperiod (light on at 8AM) and were provided food and water ad libitum.

Plasmids. The plasmids for capsid screening were constructed as follows. The pAAV-CMV-mScarlet-ΔCap-DIO-SV40pAplasmid contains an mScarlet expression cassette, an in cis Cap cassette, and a DIO cassette. The mScarlet expression cassette consists of a CMV promoter, the mScarlet coding sequence, and a SV40pA sequence. The in cis Cap cassette includes the AAV5 p41 promoter sequence, the AAV2 rep splicing sequence, and the AAV9 cap sequence. The AAV9 cap sequence was modified to introduce XbaI and AgeI sites for subsequent library generation. The DIO cassette contains a SV40pA sequence. The pCRII-9Cap-xE plasmid and the AAV2/9 REP-AAP helper plasmid were constructed following the original report. For the convenience of the construction of the library, the amino acid lysine at position 449 of the wide-type VP1, position 312 of the wide-type VP2 and/or position 247 of the wide-type VP3 was mutated to arginine.

The pAAV-SFFV backbone was constructed by replacing the Efla promoter of the pAAV-DIO-hChR2(H134R)-mCherry (Addgene 20297) with the SFFV promoter from the pHR-SFFV plasmid (Addgene 46911). The mScarlet coding sequence was subcloned into the DIO cassette to make the pAAV-SFFV-DIO-mScarlet plasmid, or was subcloned after the SFFV promoter to make the pAAV-SFFV-mScarlet plasmid. To make the pAAV-SFFV-DIO-jGCaMP8s plasmid, the jGCaMP8s (Addgene 162380) coding sequence was synthesized and subcloned into the DIO cassette. The pAAV-U6-sgRNA-SFFV-DIO-mScarlet plasmid was constructed by replacing the hSyn-Cre-2A-GFP-KASH cassette on the original plasmid (Addgene 60231) with the DIO-mScarlet cassette. The sgRNAs targeting Tmem119 (5′-GGGACCCCGTACCTTCAGCG) and Cd68 (5′-ATCCTATACCCAATTCAGGG) were selected from the mouse Brie CRISPR KO sgRNA library (Addgene 73632), and subsequently synthesized and cloned into the pAAV-U6-sgRNA-SFFV-DIO-mScarlet plasmid. The SaCas9 sgRNAs targeting P2ry12 (5′-CGGCTCCCAGTTTAGCATCACT) were designed using the web tool Benchling (https://benchling.com/crispr), and subsequently synthesized and cloned into the original pX601 plasmid45 following the SaCas9 user manual.

To minimize the potential spontaneous recombination during bacterial growth, Stbl3 cell lines were used to amplify AAV vectors that contain DIO cassettes.

AAV packaging. AAV vectors were packaged according to the protocol commonly used in the art. Briefly, the AAV vectors and the AAV helper plasmids were co-transfected into HEK293T cells. Cells were harvested 96 hours after transfection, and the viral particles were released from cells by freeze-thaw cycles and sonication. The virus was purified using cesium chloride density-gradient ultracentrifugation and dialyzed into phosphate-buffered saline (PBS) buffer. The viral titer was determined by qPCR.

Mouse microglia isolation and culture. Primary mouse microglia cells were obtained from P1 C57BL/6 wild-type mice. Pups were placed on ice for 1-2 mins until unresponsive, then were soaked with 75% alcohol, and were carefully decapitated. Brains were collected with clean sterile scissor and placed in a 10-cm dish containing 10 mL iced dissociation medium [DMEM/F12 (11330032, Gibco) supplemented with 100 U/mL penicillin and 100 μg/mL streptomycin (P/S, 15140-122, Gibco)]. All meninges were removed using No. 5 Dumont forceps under dissecting microscope. Brains were mechanically dissociated in dissociation medium. Dissociated cells were filtered through a 40-μm cell strainer and centrifuged at 1000 rpm for 10 mins at room temperature. Pellets were resuspended with culture medium [DMEM/F12 supplemented with 10% fetal bovine serum (FBS, 0099-141, Gibco), 5 ng/mL granulocyte-macrophage colony-stimulating factor (GM-CSF, PRP100489, Abbkine) and 1% P/S], and plated at a density of five brains per T-75 plastic culture flask (Falcon) pre-coated with poly-L-lysine (P8920, Sigma-Aldrich). The culture medium was changed 24 hours after isolation. After that, 50% culture medium was changed every 3 days. Two weeks later, the flasks were shaken at 180 rpm using an orbital shaker for 2 hours at 37° C. to harvest microglia. Cultured microglia were maintained at 37° C. in a humidified incubator with 5% CO₂.

In vitro AAV transduction. For testing candidate capsids identified from in vitro screening, microglia were plated in 96-well cell culture plate (6005550, PerkinElmer). Microglia were transduced with rAAVs packaged using candidate capsids at multiplicity of infection (MOI) of 10,000. Doxorubicin (0.1 μg/mL; D1515, Sigma-Aldrich) was added in the medium before rAAV transduction. After 2 days, the culture medium was changed into the TIC medium [DMEM/F12 supplemented with 1% P/S, 2 mM L-glutamine (25030-081, Gibco), 5 mg/mL N-acetyl cysteine (A9165, Sigma-Aldrich), 5 mg/mL insulin (I0516, Sigma-Aldrich), 100 mg/mL apo-transferrin (T1147, Sigma-Aldrich), 100 ng/mL sodium selenite (S5261, Sigma-Aldrich), 2 ng/mL recombinant murine TGF-02 (50153-M08H, Sino Biological), 100 ng/mL recombinant murine IL-34 (50055-M08H, Sino Biological), and 1.5 mg/mL cholesterol (ovine wool, 700000P, Merck)]. Fluorescence imaging was performed and analyzed using Opera Phenix High Content Screening System (PerkinElmer) 5 days after rAAVs transduction.

Cultured microglia RNA sequencing. For M1 microglia polarization, mouse primary microglia were exposed to 200 ng/mL lipopolysaccharide (LPS, L4130, Sigma-Aldrich) in TIC medium for 24 hours. For M2 microglia polarization, mouse primary microglia were exposed to 20 ng/mL recombinant murine interleukin-4 (214-14, PeproTech) for 24 hours. Total RNAs of untreated control, LPS-treated, interleukin-4-treated, and rAAVs-transduced microglia were extracted using Trizol (15596018, Thermo Fisher Scientific) and subjected to single-end 75 bp high-throughput sequencing on an Illumina platform.

In vitro screening. The detail sequences of primers used in this study are listed in Table 1. AAV-cMG.WPP and AAV-cMG.QRP were identified by screening for AAV variants that effectively transduced cultured mouse microglia. An AAV capsid library was first constructed by inserting random heptamers into the reading frame for each capsid protein, VP 1-3, of the AAV9 capsid using the CREATE protocol. Briefly, the library fragments were generated by PCR using the XF and 7xMNN primers with the pCRII-9Cap-xE plasmid serving as the template. The pAAV-CMV-mScarlet-ΔCap-DIO-SV40pA plasmid was linearized by XbaI and AgeI. The library fragments were assembled into the linearized the pAAV-CMV-mScarlet-ΔCap-DIO-SV40pA plasmid using Gibson assembly. The resulted library was packaged into rAAVs by co-transfecting the AAV capsid library, the AAV2/9 REP-AAP helper plasmid and the AAV-helper plasmid into HEK293T cells. Approximately 10 library rAAVs were used to transduce the cultured mouse microglia for 24 hours. 48 hours after transduction, the genomes of rAAVs that had successfully transduced the cultured microglia were recovered using Trizol. The cap sequences were first amplified from recovered AAV genomes by PCR using used the 9CapF and SV40pA-R primers. The PCR product was purified and used as the template for the second PCR reaction that used the XF and 588i-R primers. The recovered cap sequences were then assembled back into the pAAV-CMV-mScarlet-ΔCap-DIO-SV40pA plasmid and screened again in the cultured mouse microglia. The candidates that were highly enriched after two rounds of screening were identified through next generation sequencing (NGS) and individually tested. The enrichment score of a variant was calculated as follows:

Enrichment score=Log₁₀((normalized read counts in round 2)/(normalized read counts in round 1)).

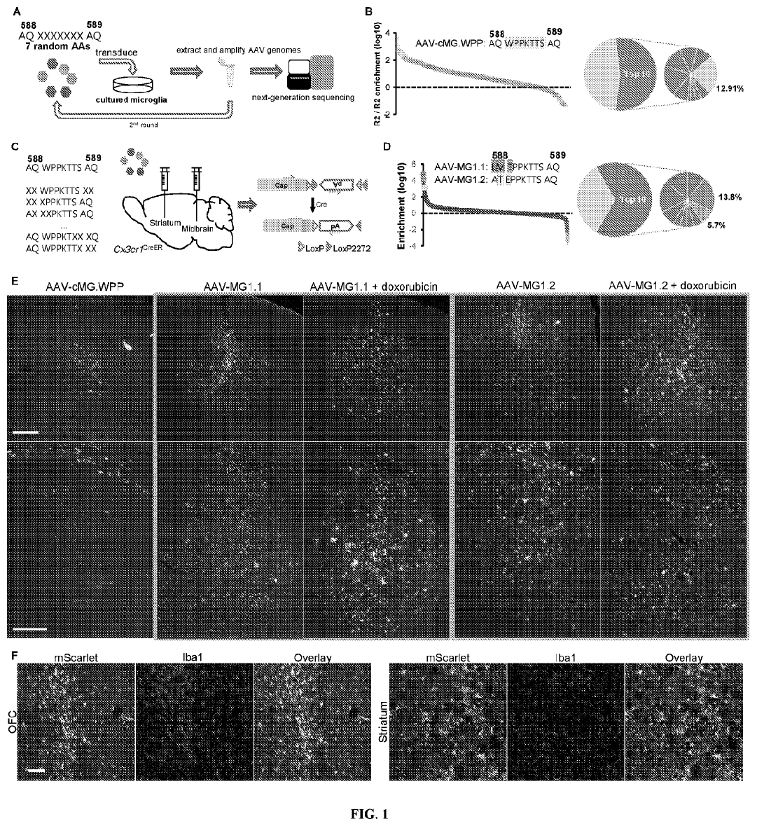

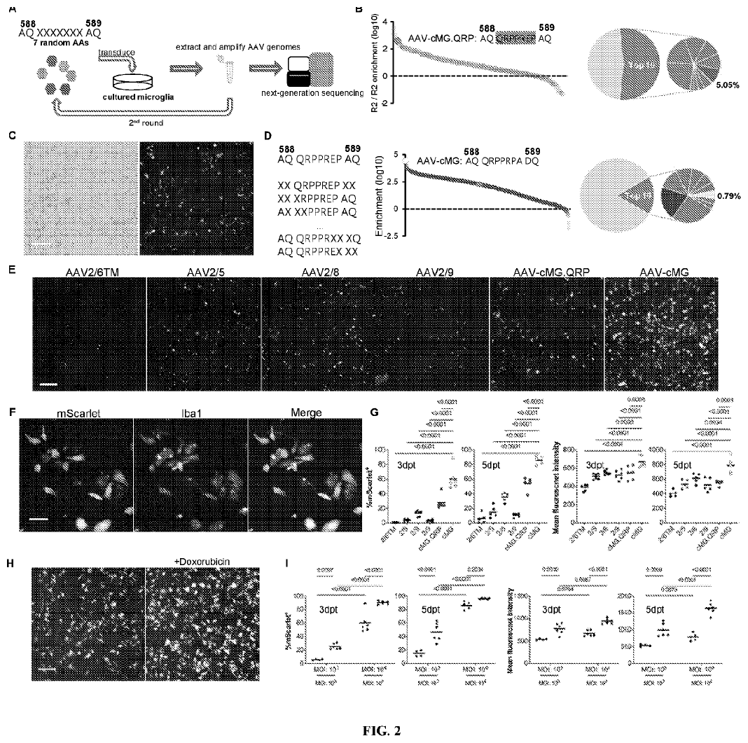

To further identify AAV-cMG.WPP and AAV-cMG.QRP variants that mediate more efficient microglial transduction in vitro, AAV-cMG.WPP and AAV-cMG.QRP capsid mutant libraries in which the inserted heptamer and the four flanking amino acids in the AAV-cMG.WPP or AAV-cMG.QRP capsids were randomized was constructed (FIGS. 1C and 2D). The AAV-cMG.WPP and AAV-cMG.QRP capsid mutant libraries were screened in cultured mouse microglia as described above.

TABLE 1
Primers sequences for library construction and variant recovery.

| Name | Sequence (5′-3′) |
|---|---|
| XF | ACTCATCGACCAATACTTGTACTATCTCTCTAGAAC (SEQ ID NO.: 12) |
| 7xMNN | GTATTCCTTGGTTTTGAACCCAACCGGTCTGCGCCTGTGCMNNMNNMNNMNN |
| | MNNMNNMNNTTGGGCACTCTGGTGGTTTGTG (SEQ ID NO.: 13) |
| 9CapF | CAGGTCTTCACGGACTCAGACTATCAG (SEQ ID NO.: 14) |
| CDF | CATTGATGAGTTTGGACAAACCACAACTAGAATG (SEQ ID NO.: 15) |
| 1527 | ACACTCTTTCCCTACACGACGCTCTTCCGATCTGACAAGTGGCCACAAACCAC |
| | CAG (SEQ ID NO.: 16) |
| 1532 | GTGACTGGAGTTCAGACGTGTGCTCTTCCGATCTCCTTGGTTTTGAACCCAACC |
| | G (SEQ ID NO.: 17) |
| SV40pA-R | CATTCTAGTTGTGGTTTGTCCAAACTCATCAATG (SEQ ID NO.: 18) |
| 588i-F | AATAGCTTGATGAATCCTGGACCTG (SEQ ID NO.: 19) |
| 588i-R | TTCCTTGGTTTTGAACCCAACCG (SEQ ID NO.: 20) |
| WPP-mut-R1 | GTATTCCTTGGTTTTGAACCCAACCGGTCTGCGCCTGTGCAGACGTAGTCTTCG |
| | GCGGMNNMNNMNNACTCTGGTGGTTTGTG (SEQ ID NO.: 21) |
| WPP-mut-R2 | GTATTCCTTGGTTTTGAACCCAACCGGTCTGCGCCTGTGCAGACGTAGTCTTCG |
| | GMNNMNNMNNGGCACTCTGGTGGTTTGTG (SEQ ID NO.: 22) |
| WPP-mut-R3 | GTATTCCTTGGTTTTGAACCCAACCGGTCTGCGCCTGTGCAGACGTAGTCTTM |
| | NNMNNMNNTTGGGCACTCTGGTGGTTTGTG (SEQ ID NO.: 23) |
| WPP-mut-R4 | GTATTCCTTGGTTTTGAACCCAACCGGTCTGCGCCTGTGCAGACGTAGTMNNM |
| | NNMNNCCATTGGGCACTCTGGTGGTTTGTG (SEQ ID NO.: 24) |
| WPP-mut-R5 | GTATTCCTTGGTTTTGAACCCAACCGGTCTGCGCCTGTGCAGACGTMNNMNN |
| | MNNCGGCCATTGGGCACTCTGGTGGTTTGTG (SEQ ID NO.: 25) |
| WPP-mut-R6 | GTATTCCTTGGTTTTGAACCCAACCGGTCTGCGCCTGTGCAGAMNNMNNMNN |
| | CGGCGGCCATTGGGCACTCTGGTGGTTTGTG (SEQ ID NO.: 26) |
| WPP-mut-R7 | GTATTCCTTGGTTTTGAACCCAACCGGTCTGCGCCTGTGCMNNMNNMNNCTT |
| | CGGCGGCCATTGGGCACTCTGGTGGTTTGTG (SEQ ID NO.: 27) |
| WPP-mut-R8 | GTATTCCTTGGTTTTGAACCCAACCGGTCTGCGCCTGMNNMNNMNNAGTCTT |
| | CGGCGGCCATTGGGCACTCTGGTGGTTTGTG (SEQ ID NO.: 28) |
| WPP-mut-R9 | GTATTCCTTGGTTTTGAACCCAACCGGTCTGCGCMNNMNNMNNCGTAGTCTT |
| | CGGCGGCCATTGGGCACTCTGGTGGTTTGTG (SEQ ID NO.: 29) |
| WPP-mut-R10 | GTATTCCTTGGTTTTGAACCCAACCGGTCTGCGCMNNMNNAGACGTAGTCTTC |
| | GGCGGCCAMNNMNNACTCTGGTGGTTTGTG (SEQ ID NO.: 30) |
| QRP-mut-R1 | gtattccttggttttgaacccaAccggtctgcgcctgtgcCGGCTCACGCGGAGGCCTMNNMNNMNNa |
| | ctctggtggtttgtg (SEQ ID NO.: 31) |
| QRP-mut-R2 | gtattccttggttttgaacccaAccggtctgcgcctgtgcCGGCTCACGCGGAGGMNNMNNMNNggcac |
| | tctggtggtttgtg (SEQ ID NO.: 32) |
| QRP-mut-R3 | gtattccttggttttgaacccaAccggtctgcgcctgtgcCGGCTCACGCGGMNNMNNMNNttgggcactct |
| | ggtggtttgtg (SEQ ID NO.: 33) |
| QRP-mut-R4 | gtattccttggttttgaacccaAccggtctgcgcctgtgcCGGCTCACGMNNMNNMNNCTGttgggcactct |
| | ggtggtttgtg (SEQ ID NO.: 34) |
| QRP-mut-R5 | gtattccttggttttgaacccaAccggtctgcgcctgtgcCGGCTCMNNMNNMNNCCTCTGttgggcactct |
| | ggtggtttgtg (SEQ ID NO.: 35) |
| QRP-mut-R6 | gtattccttggttttgaacccaAccggtctgcgcctgtgcCGGMNNMNNMNNAGGCCTCTGttgggcactct |
| | ggtggtttgtg (SEQ ID NO.: 36) |
| QRP-mut-R7 | gtattccttggttttgaacccaAccggtctgcgcctgtgcMNNMNNMNNCGGAGGCCTCTGttgggcactct |
| | ggtggtttgtg (SEQ ID NO.: 37) |
| QRP-mut-R8 | gtattccttggttttgaacccaAccggtctgcgcctgMNNMNNMNNACGCGGAGGCCTCTGttgggcact |
| | ctggtggtttgtg (SEQ ID NO.: 38) |
| QRP-mut-R9 | gtattccttggttttgaacccaAccggtctgcgcMNNMNNMNNCTCACGCGGAGGCCTCTGttgggca |
| | ctctggtggtttgtg (SEQ ID NO.: 39) |
| QRP-mut-R10 | NNactctggtggtttgtg (SEQ ID NO.: 40) |
| | gtattccttggttttgaacccaAccggtctgcgcMNNMNNCGGCTCACGCGGAGGCCTCTGMNNM |

In vivo screening. To identify AAV-cMG.WPP and AAV-cMG.QRP variants that effectively transduced mouse microglia in vivo, a AAV-cMG.WPP and AAV-cMG.QRP capsid mutant libraries in which the inserted heptamer and the four flanking amino acids in the AAV-cMG.WPP capsid were randomized was constructed (FIGS. 1C and 2D). Briefly, the library fragments were generated by ten separated PCR reactions using the XF and WPP-mut-R1-10 primers with the pCRII-9Cap-xE plasmid serving as the template. Equal amounts of ten PCR products were mixed and assembled into the pAAV-CMV-mScarlet-ΔCap-DIO-SV40pA plasmid using Gibson assembly. The resulted library was packaged into rAAVs as described above. The AAV-cMG.WPP and AAV-cMG.QRP capsid mutant library rAAVs were injected bilaterally into the striatum (800 nL) and the midbrain (500 nL) of three Cx3cr1^{CreER} mice. Tamoxifen was injected (i.p., 10 mg/kg) for five consecutive days following virus injection. Mice were sacrificed ten days after virus injection. The brains were dissected, and the genomes of rAAVs that have successfully transduced cells in vivo were recovered using Trizol. The cap sequences in the Cre-recombined genomes were selectively amplified using the 9CapF and CDF primers. The candidates that were highly enriched were identified through NGS and individually tested. The enrichment score of a variant was calculated as follows:

Enrichment score=Log₁₀((normalized read counts in in vivo screened sample)/(normalized read counts in the AAV library)).

Screening NGS sample preparation. Library rAAVs were treated with proteinase K at 37° C. overnight. The AAV genomes were obtained by phenol chloroform extraction and ethanol precipitation. 100 ng AAV genomes were used as the PCR template. The cap fragments that contain the inserted heptamers were amplified using the 588i-F and 588i-R primers. The PCR products were purified and used as the template for the second PCR using the 1527 and 1532 primers. A final PCR was performed to add unique indices for subsequent NGS using standard indexed primers. The indexed PCR products were size selected by 2% agarose gel before submitting to NGS.

For in vivo selection, the cap sequences in the Cre-recombined AAV genomes were selectively amplified using the 9CapF and CDF primers. The cap fragments that contain the inserted heptamers were then amplified and indexed as described above.

Common surgery and virus injection. Mice were anaesthetized with pentobarbital (i.p., 80 mg/kg) before surgery, and then placed in a mouse stereotaxic instrument. Then the virus was injected in Orbitofrontal cortex (OFC), striatum, midbrain, hippocampus, or thalamus of the mice.

Injections were performed using a microsyringe pump (Nanoliter 2010 Injector, WPI) and a Micro4 controller (WPI). The virus was delivered to the target areas at a rate of 46 nL/min. Doxorubicin (150 ng/g) was injected retro-orbitally immediately after virus injection.

All subsequent experiments were performed at least 2 weeks after virus injection, except for those involved in vivo CRISPR/Cas9 KO which were performed at least 3 weeks after virus injection. For Cx3cr1^{CreER} mice, tamoxifen was i.p. injected for 3 consecutive days (5 consecutive days for the in vivo screening described above) after virus injection.

Single cell isolation for microglia single-cell RNA sequencing (scRNA-seq). To minimize ex vivo activation, a cold-mechanical dissociation protocol was employed. All procedures were performed on ice with cold buffers or in refrigerated centrifuge. Mice were deeply anesthetized and perfused. Brains were quickly removed and immersed in Dounce buffer (HBSS with HEPES+DNase+RNase inhibitor) and cut into smaller chunks. The tissue solution was quickly transferred to a 15 ml Dounce homogenizer and gently homogenized with a loose-fitting pestle for ˜10 times. The remaining tissue pieces were allowed to sediment and the supernatant containing cell suspensions were collected to a new tube. New Dounce buffer was added to the sediment tissue and the homogenization was repeated for another round. The collected cell solution was centrifuged, resuspended, and passed sequentially through 70-μm and 30-μm pre-wet cell strainers to remove debris. The cells were centrifuged once more and resuspended in 37% stock isotonic Percoll (SIP). A Percoll gradient of HBSS/30%/37%(cells)/70% was used to enrich microglia by centrifugating at 200 g for 20 mins with minimal acceleration and no brake. Cells in the interphase between 30% and 37% were carefully collected, washed, and resuspended in 0.04% BSA in Dulbecco's PBS. Cells were then manually picked for Smart-seq or run through the 10′ Genomics Chromium Single Cell 3′ v3 protocol, and were subsequently sequenced on the Illumina platform.

Smart-seq2-based scRNA-seq library construction and sequencing. Single microglia cells in Bovine Serum Albumin (BSA) buffer [0.04% BSA (0332, Amresco) in HBSS buffer] were picked under stereo fluorescence microscope using a mouth pipette. The library construction was conducted using a modified STRT-seq protocol to construct the single-cell RNA-seq library as previously described39. Briefly, cells were lysed, and mRNAs were released into the lysis buffer containing barcoded reverse transcription primers. mRNAs were reverse transcribed into cDNA. After cDNA pre-amplification, single-cell transcriptomes tagged with different barcodes were pooled together. Additional 4 cycles of PCR were performed using biotin-modified primers in order to enrich the 3′ end of cDNAs. The amplicons were randomly sheared into ˜300 bp fragments by sonication (Covaris) and purified using Dynabeads MyOne Streptavidin C1 (65002, Invitrogen). The purified fragments were then processed on the Illumina platform for sequencing of 150 bp pair-end reads.

Sequencing data processing and quality control (QC). Reference datasets generated by 10′ Genomics platform were aligned to a pre-built GRCm38 reference genome by ‘cellranger count’ and combined by ‘cellranger aggr’ commands (Cell Ranger v3.1.0). The resulting feature-barcode-matrix files was loaded with Seurat41 (v4.0.2) and filtered with the following QC criteria: remove cells with mitochondrial RNA greater than 10% and cells expressing more than 300 features, and remove features expressed in less than 10 cells. After QC, Seurat workflow was ran with default parameters and retained bona fide microglia from the dataset by manually subsetting clusters showing consistently high expression of Hexb58, using the CellSelector function over the UMAP embedding.

For the Smart-seq2 dataset, a protocol listed in the umi-tools documentation (https://umi-tools.readthedocs.io/en/latest/Single_cell_tutorial.html) was adopted, in which the fastq files were demultiplexed with UMI-tools59, aligned with STAR60 (v2.5.4b) and quantified with featureCounts61 (Subread v1.6.3). A minor modification was made to the ‘umi_tools whitelist’ procedure, where 100 cells were searched to obtain an extended whitelist, but ‘washed’ the whitelist by retaining only those cell barcodes actually used in library preparation steps, which was named as “ground truth barcodes”. In the “umi_tools extract” step, only those reads containing the “ground truth barcodes” were kept, whereas reads with adaptor contaminations or major sequencing errors were removed. For quality control, the library size, feature number, mitochondrial RNA percentage and Ribosomal RNA percentage from the feature-cell matrix were computed. The scater62 (v1.18.6) quickPerCellQC function was used to identify and remove cell outliers based on these four metrics. The transduction of rAAVs in sequenced microglia was determined by the detection of the mScarlet reporter transcript.

Unsupervised analysis for dimension reduction and identification of microglia states. Seurat workflow was performed with default parameters on both the 10′ reference dataset and the Smart-seq2 dataset. Standard PCA for both datasets was computed and the dimension of the datasets was chosen as 40 (dims=1:40). To identify microglia states, the reference dataset was first annotated by labeling microglia from control mice as “Homeostatic” and microglia from LPS-treated mice as “Reactive”. Then, the transfer anchors were found between the query (Smart-seq2) and the reference (10′) datasets in the PCA space with FindTransferAnchors function, and projected the query data onto the reference dataset with MapQuery function. The reference and query datasets were further merged, and UMAP dimension reduction was re-run on the merged dataset to obtain a ‘de novo visualization’ in the UMAP space.

In vivo two-photon imaging. rAAVs (1 μL) packaged using the AAV-MG1.1 or AAV-MG1.2 capsids were injected into the layers II-IV of the primary somatosensory cortex (S1) of Cx3cr1^{CreER} or Cx3cr1GFP mice at the age of 8 weeks. After two weeks, a circular skull 3 mm in diameter centered over the virus injection site was carefully removed. A custom-designed steel head bar that included an imaging chamber was positioned over the craniotomy and affixed to the exposed skull with cyanoacrylate glue and dental cement. Mice were allowed to recover from the surgery for one week. Two-photon imaging was performed 100-150 μm below the dura mater using a FluoView FVMPE-RS microscope (Olympus, 25×, 1.05 NA water-immersion lens). The laser was tuned to 920 nm, and was maintained below 50 mW for jGCaMP8s and GRAB_{ATP} imaging or below 40 mW for GFP imaging. Microglia were imaged using z-stacks, each of which consisted of 16 images spaced 2 μm apart (30 μm total depth). Lateral shifts were corrected using the StackReg plugins. Data quantification was conducted using customized Matlab scripts and the GraphPad Prism software.

To monitor jGCaMP8s and GRAB_{ATP1,0} fluorescence signal changes following LPS challenge, a 636×636 μm field of view (512×512 pixel resolution, 1.24 μm/pixel) was imaged at 1.5 fps. A 10-min time-lapse imaging session (50 z-stacks) was first performed to record the baseline fluorescence. LPS (10 mg/kg) was then i.p. injected. Immediately following LPS injection, additional 10-min imaging sessions (6 sessions for jGCaMP8s imaging and 7 sessions for GRAB_{ATP1,0} imaging) were performed at 50-min intervals. A maximum projection was created for each stack. Microglia somata were manually selected. For a 10-min imaging session, the fluorescence intensity of a microglia soma was averaged across 25 z-stacks.

To monitor GRAB_{ATP1,0} fluorescence signal changes following laser ablation, a 636×636 μm field of view (512×512 pixel resolution) was imaged at 1.5 fps. A circular area of 15 μm in diameter in the center of imaging field was focused, and 70 pulses of 920 nm laser (30 ms per pulse) at ˜500 mW were applied to induce acute tissue damage. Immediately following laser ablation, 40-min time-lapse imaging (200 z-stacks) was performed. A maximum projection was created for each stack. Microglia somata were manually selected to calculate the fluorescence intensity.

For imaging responses of GFP-expressing microglia towards acute tissue damage, a 318×318 μm field of view (512×512 pixel resolution, 0.62 μm/pixel) was imaged at 1.5 fps. The acute tissue damage was induced as described above. Immediately following laser ablation, 40-min (control mice; 200 z-stacks) or 80-min (P2ry12 knockout mice; 400 z-stacks) time-lapse imaging was performed. The normalized microglial response was computed as described4.

Immunohistochemistry. Mice were anesthetized with an overdose of pentobarbital and perfused intracardially with PBS, followed by paraformaldehyde (PFA, 4% wt/vol in PBS). Brains were dissected and postfixed in 4% PFA for at least 4 hours at room temperature. Samples were then dehydrated in 30% sucrose solution. Brain sections (30 μm) were prepared on a Cryostat microtome (Leica CM1950). Sections were permeabilized with 0.3% Triton X-100 in PBS (PBST) and blocked in 2% BSA in PBST at room temperature for 1 hour. Sections were then incubated with primary antibodies (anti-Ibal, 1:500, 019-19741, Wako; anti-TMEM119, 1:1000, ab209064, Abcam; anti-CD68, 1:500, ab53444, Abcam; anti-P2RY12, 1:100, 848002, BioLegend) overnight at 4° C. Samples were washed three times in PBST and were then incubated with fluorescent secondary antibodies (Goat anti-rabbit-AF647, 111-605-144, Jackson ImmunoResearch; Goat anti-rabbit-AF488, 111-545-003, Jackson ImmunoResearch; Goat anti-rat-Cy5, 112-175-143, Jackson ImmunoResearch) at room temperature for 2 hours.

For cultured microglia, cells were first washed in cold PBS and then fixed in 4% PFA for 10 min at room temperature. After washed again in PBS, cells were permeabilized in PBST and blocked in 2% BSA in PBST at room temperature for 20 min. Cells were then incubated with antibodies (anti-Ibal, 1:500, 019-19741, Wako) at room temperature for 2 hours. Cells were washed three times in PBST and were then incubated with fluorescent secondary antibodies (Goat anti-rabbit-AF488, 111-545-003, Jackson ImmunoResearch) at room temperature for 1 hour.

Image acquisition and analysis of fixed tissues. Confocal microscopy was performed on a Zeiss LSM880 confocal scanning microscope. For slide scanner imaging, the wide-field fluoresce imaging was performed by using the Olympus VS120 virtual microscopy slide scanning system with a 10′ objective. Cell counting was performed using Imaris. To assess the effect of doxorubicin administration on mScarlet expression, Imaris was used to measure the mean fluorescent intensity of labeled cells in the striatum on 9 brain sections centered on the virus injection site. To quantify the knockout efficiency of the Tmem119 sgRNA, the percentage of signal-positive pixels was calculated over an area of 1.5 mm×1.5 mm that covers the dorsal striatum using the “Threshold” function in ImageJ. To quantify the knockout efficiency of the P2ry12 sgRNA, the percentage of signal-positive pixels was calculated over an area of 1 mm×1 mm that covers the S1 cortex using the “Threshold” function in ImageJ. For Cd68 knockout experiments, the number of Cd68-positive cells was quantified in an area of 1.5 mm×1.5 mm that covers the dorsal striatum using Imaris.

### EXAMPLES

### Example 1. Screen of the Capsid Library In Vitro

The wildtype AAV9 capsid was used as the starting point for generating a capsid library, in which each AAV9 capsid variant harbors a random seven-amino-acid insertion between amino acids 588 and 589 of the AAV9 VP1 protein (FIG. 1A). This library was packaged into rAAVs and screened in cultured mouse microglia for two consecutive rounds (FIGS. 1A and 2A). The cultured mouse microglia were transduced with the capsid library rAAVs and the capsid variants that have successfully mediated transduction were recovered. Then, the recovered capsid variants were packaged into rAAVs and screened again in cultured mouse microglia. By next-generation sequencing, the capsid variants that were highly enriched after two rounds of screening were identified (FIGS. 1B and 2B).

Two capsid variants, one harbors a “WPPKTTS” heptamer insertion (hereinafter referred to as AAV-cMG.WPP; FIG. 1B) and one harbors a “QRPPREP” heptamer insertion (hereinafter referred to as AAV-cMG.QRP; FIG. 2B), showed significantly higher transduction of cultured microglia, as compared to the other candidates tested. The VP1 protein of AAV-cMG.WPP has an amino acid sequence as shown by SEQ ID NO.: 7, and the VP1 protein of AAV-cMG.QRP has an amino acid sequence as shown by SEQ ID NO.: 8. Then, single-stranded mScarlet reporter vectors were packaged into rAAVs using candidate capsid variants, respectively, and were transduced into cultured mouse microglia. The transduction abilities of the capsid variants were evaluated as compared to the parental AAV9 capsid, as well as three AAV capsids [AAV5, AAV8, and AAV6 with Y731F/Y705F/T492V triple mutation (AAV6TM)28] that have been reported to transduce cultured mouse microglia.

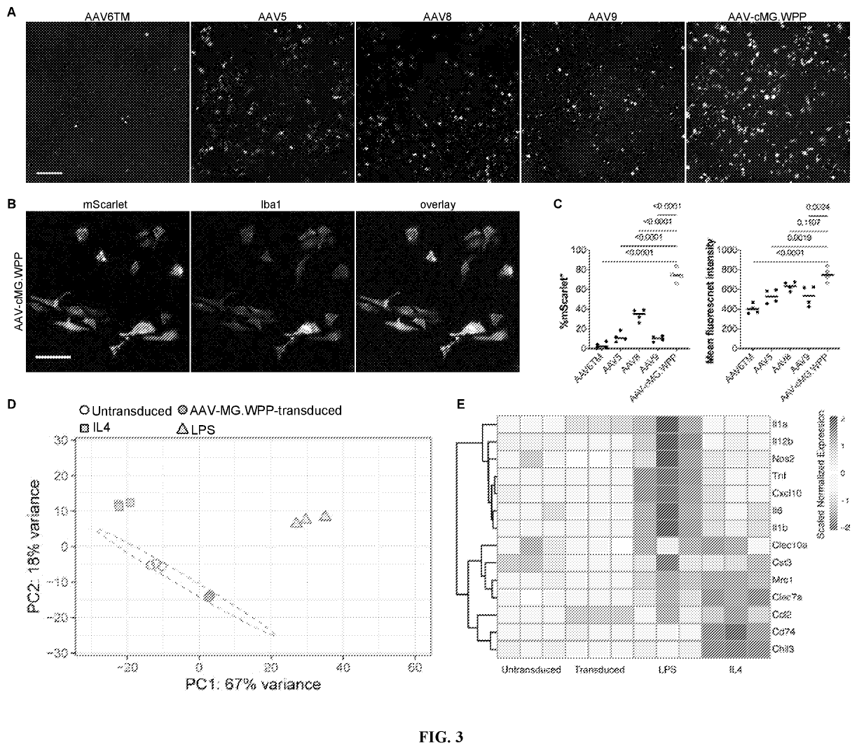

AAV-cMG.WPP was enriched over 170-fold and made up 12.91% of the total recovered variants in the second round of screening (FIG. 1B). Dramatically higher transduction rate was achieved by AAV-cMG.WPP (˜75%) as compared with that by the AAV5 (˜12%), AAV6TM (˜3%), AAV8 (˜34%), or AAV9 (˜10%) capsid (FIGS. 3A-C). AAV-cMG.WPP also drives significantly stronger mScarlet expression than that by the AAV5, AAV6TM, AAV8, or AAV9 capsid (FIGS. 3A and C).

AAV-cMG.QRP was enriched ˜400-fold and made up 5.05% of the total recovered variants in the second round of screening (FIG. 2B). Significantly higher transduction rate and stronger mScarlet expression was achieved by AAV-cMG.QRP as compared with that by the AAV5, AAV6TM, AAV8, or AAV9 capsid (FIGS. 2C, E, and G).

### Example 2. Examination of the Influence of AAV-cMG.WPP on Microglia Phenotype

External stimuli can induce microglia into the reactive state or the alternative activation state. To examine whether AAV-cMG.WPP-mediated transduction can trigger microglia phenotype changes, a principal component analysis was performed for the transcriptomes data obtained from four different samples: control untransduced (homeostatic state), LPS-treated (reactive state), Interleukin-4-treated (alternative activation state), and AAV-cMG.WPP-transduced cultured mouse microglia (FIG. 3D). AAV-cMG.WPP-transduced microglia clustered towards control untransduced microglia, and away from LPS-treated or Interleukin-4-treated microglia (FIG. 3D). Further differential gene expression analysis also indicated that AAV-cMG.WPP transduction did not induce the proinflammatory pathways in cultured microglia (FIG. 3E). Thus, these results demonstrate the utility of AAV-MG.WPP to mediate safe transgene delivery in cultured microglia.

### Example 3. Further Screening of a Semi-Randomly Mutating Capsid Library from AAV-cMG.WPP for In Vivo Transduction

An additional capsid library was generated by semi-randomly mutating the inserted heptamer and the adjacent four amino acids in AAV-cMG.WPP (FIG. 1C). This new library was packaged into rAAVs and screened in vivo by injecting the library rAAVs into the brains of Cx3cr1^{CreER} mice. The CREATE strategy was adopted to selectively recover capsid variants from Cre-recombined AAV genomes (i.e., genomes of rAAVs that have successfully transduced microglia in vivo). After two rounds of screening, two highly enriched capsid variants were identified (FIG. 1D), both of which contain mutations at amino acid positions 587-589 of AAV-cMG.WPP. The first variant comprises the amino acid sequence “LMT” at positions 587-589 and accounts for 13.8% of the total recovered variants (FIG. 1D). The second variant comprises the amino acid sequence “ATE” at positions 587-589 and account for 5.7% of the total recovered variants (FIG. 1D). These two AAV-cMG.WPP capsid variants were named as AAV-MG1.1 and AAV-MG1.2, respectively. The VP1 protein of AAV-MG1.1 has an amino acid sequence as shown by SEQ ID NO.: 10, and the VP1 protein of AAV-MG1.2 has an amino acid sequence as shown by SEQ ID NO.: 11.

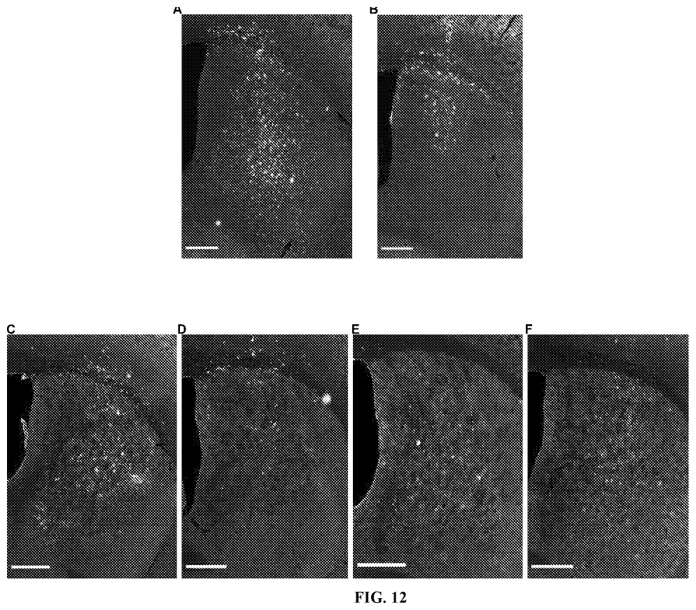

Four additional AAV-cMG.WPP variants were also identified to be capable of transducing microglia in vivo and inducing strong and widespread mScarlet expression in the striatum of Cx3cr1^{CreER} mice (FIGS. 12C-F). The first variant, AAV-MG.PTS, comprises the amino acid sequence “PTS” at positions 589-591 of AAV-cMG.WPP (FIG. 12C). The second variant, AAV-MG.LMV, comprises the amino acid sequence “LMV” at positions 589-591 of AAV-cMG.WPP (FIG. 12D). The third variant, AAV-MG.WTD, comprises the amino acid sequence “WTD” at positions 589-591 of AAV-cMG.WPP (FIG. 12E). The fourth variant, AAV-MG.VLS, comprises the amino acid sequence “VLS” at positions 588-590 of AAV-cMG.WPP (FIG. 12F). The VP1 protein of AAV-MG. PTS has an amino acid sequence as shown by SEQ ID NO.: 59. The VP1 protein of AAV-MG. LMV has an amino acid sequence as shown by SEQ ID NO.: 62. The VP1 protein of AAV-MG.WTD has an amino acid sequence as shown by SEQ ID NO.: 65. The VP1 protein of AAV-MG.VLS has an amino acid sequence as shown by SEQ ID NO.: 68.

### Example 4. Examination of the Ability of AAV-MG1.1 and AAV-MG1.2 to Transduce Microglia In Vivo

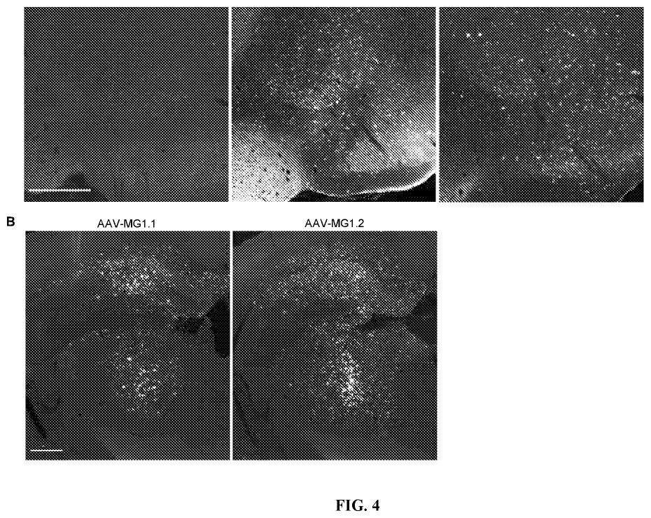

AAV-MG1.1 and AAV-MG1.2 were used to package a single-stranded Cre-dependent mScarlet reporter vector into rAAVs (AAV-MG1.1/1.2-SFFV-DIO-mScarlet). Then, these rAAVs were injected into the brains of Cx3cr1^{CreER} mice and evaluated against the corresponding AAV-cMG.WPP rAAVs. Both AAV-MGs drove strong and widespread mScarlet expression in all brain areas tested (the orbitofrontal cortex, the striatum, the midbrain, the hippocampus, and the thalamus; FIGS. 1E, 4A, 4B and 5A).

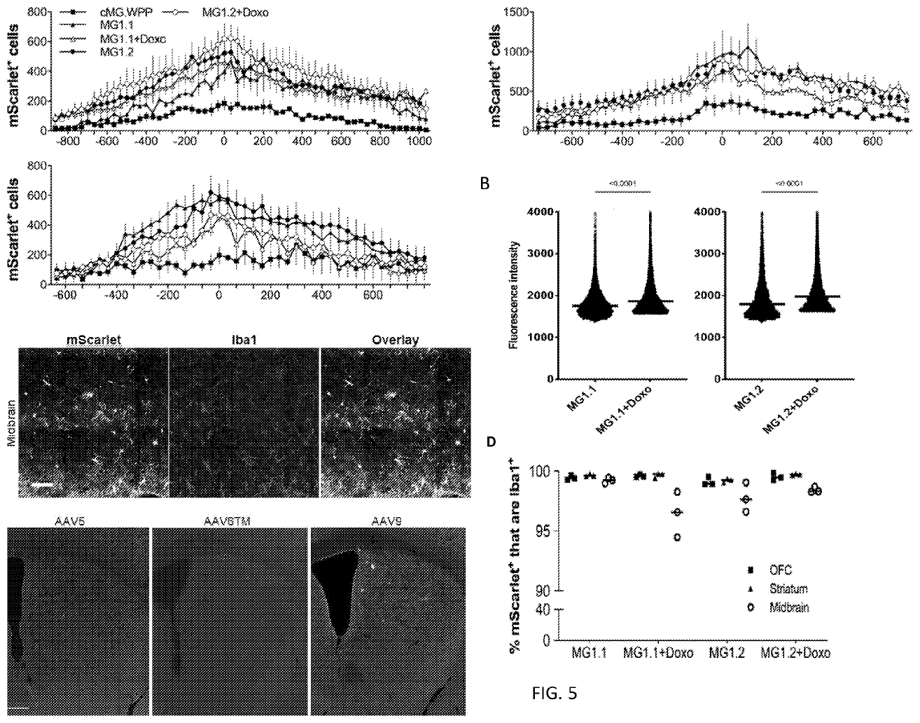

Further, it was found by using the immunoreactivity of ionized calcium binding adaptor molecule 1 (Ibal) as the marker of microglia that the mScarlet expression was restricted in microglia and that nearly all microglia in the core regions of the injection sites were labeled (FIGS. 1F and 5C and 5D). The in vivo microglial transduction efficiency of both AAV-MGs is also much higher than that of the parental AAV9 capsid as well as than that of the AAV5 and AAV6TM capsids. Only sparse and dim labeling of microglia was achieved by AAV9, AAV5, or AAV6TM rAAVs in Cx3cr1^{CreER} mice (FIG. 5E).

### Example 5. Examination of the Influence of Topoisomerase Inhibitor on the In Vivo Microglial Transduction Efficiency of AAV-MGs

Previous studies have established that inhibiting topoisomerases and proteasomes using small-molecule drugs facilitates rAAV transduction, both in vitro and in vivo. Therefore, it was examined whether this approach may further enhance the in vivo microglial transduction efficiency of AAV-MGs. An FDA-approved topoisomerase inhibitor doxorubicin, which has been shown to increase rAAV expression level in neurons in vivo, was used for this examination. Immediately following the stereotaxic intracranial injection of AAV-MG1.1/1.2-SFFV-DIO-mScarlet in Cx3cr1^{CreER} mice, doxorubicin was administrated via retro-orbital injection. For both AAV-MGs, doxorubicin administration significantly enhanced the mScarlet expression level in microglia but did not increase the number of mScarlet-labeled microglia (FIGS. 1E, 5A and 5B). These results demonstrate the possibility of further enhancing AAV-mediated transgene expression in microglia using pharmacological approaches.

### Example 6. Examination of the Influence of AAV-MGs-Mediated Transduction on the In Vivo Activation of Microglia

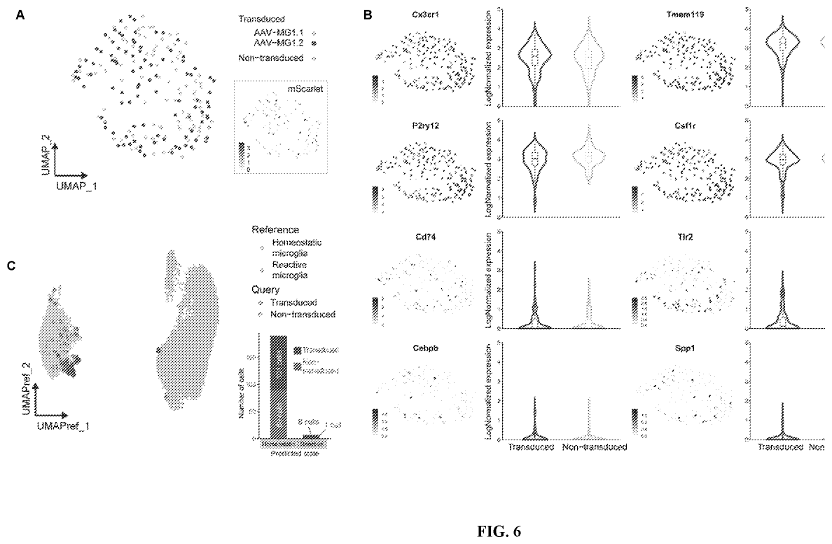

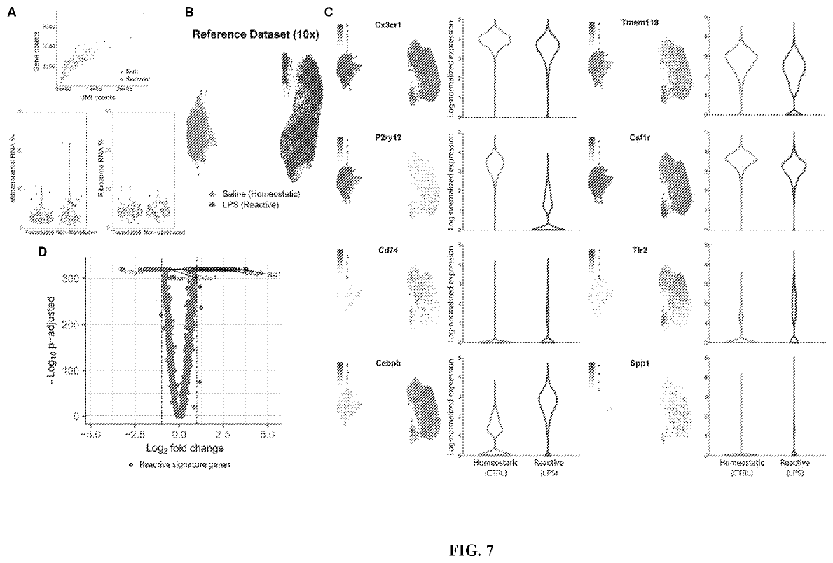

After establishing that AAV-MGs mediate efficient transduction of microglia in vivo, it was examined whether AAV-MGs-mediated transduction would trigger microglia phenotype changes in the brain. Two weeks after the injection of the AAV-MG1.1/1.2-SFFV-DIO-mScarlet into the striatum of Cx3cr1^{CreER} mice, both mScarlet⁺ and mScarlet-microglia were selected for single-cell RNA sequencing (scRNA-seq) using a modified Smart-seq2 protocol. Projection of the microglia using uniform manifold approximation and projection (UMAP) indicated that the non-transduced and transduced microglia formed a homogeneous cluster (FIGS. 6A and 7A). Moreover, there was no difference in the expression of homeostatic microglia marker genes or reactive microglia marker genes between the non-transduced and transduced microglia (FIG. 6B). A label transfer strategy implemented in Seurat was leveraged to project microglia from the Smart-seq2 dataset onto the reference 10× datasets of homeostatic and reactive microglia (FIGS. 7B-D), and recomputed a merged UMAP projection to represent the microglia states from both datasets (FIG. 6C). The majority of microglia in the Smart-seq2 dataset was positioned within the homeostatic microglia cluster (FIG. 6C). These results support that the transduction mediated by AAV-MGs does not induce microglia activation in vivo.

### Example 7. Analysis of the Delivery Efficiency of AAV-MGs into Microglia

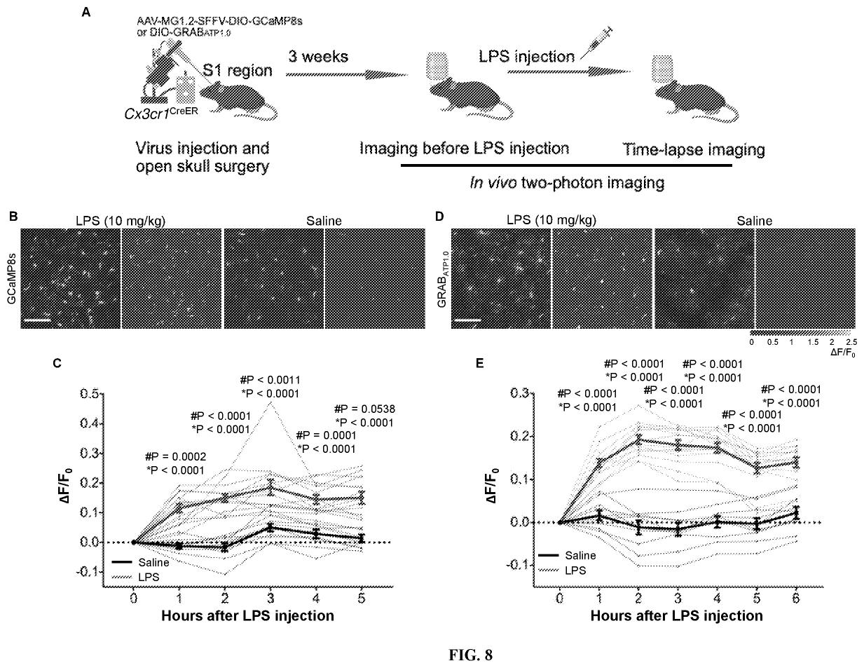

In this example, it was tested whether AAV-MGs could efficiently deliver various genetic payloads into microglia. Two newly developed genetically-encoded fluorescent sensors in microglia were expressed to examine the physiological responses of microglia to peripheral endotoxin challenges (FIG. 8A). First, AAV-MG1.2 was used to package a single-stranded Cre-dependent AAV vector that bears the newest generation calcium indicator jGCaMP8s (AAV-MG1.2-SFFV-DIO-GCaMP8s). The rAAVs were injected into the primary somatosensory (SI) cortex of Cx3cr1^{CreER} mice. After two weeks allowing viral transgene expression, 10 mg/kg LPS were injected (i.p.) to induce systemic inflammation. The Ca²⁺ signals in microglia somata were tracked by means of two-photon imaging (FIG. 8A). The results showed a significant increase of Ca²⁺ signals at one hour after LPS injection (FIGS. 8B and 8C). This LPS-induced elevation of intracellular Ca²⁺ was sustained and peaked at three hours post-injection, and then started to decrease (FIGS. 8B and 8C). The increase of Ca²⁺ signals was not caused by the i.p. injection procedure per se, since the i.p. injection of saline did not lead to significant jGCaMP8s fluorescent changes (FIGS. 8B and 8C).

Further, a single-stranded Cre-dependent AAV vector was packaged to contain the newly developed ATP fluorescent sensor GRAB_{ATP1,0} using AAV-MG1.2 (AAV-MG1.2-SFFV-DIO-GRAB_{ATP1,0}). The GRAB_{ATP1,0} sensor was expressed in the microglia in the Si cortex of Cx3cr1^{CreER} mice and the GRAB fluorescence signals were monitored by means of in vivo two-photon imaging. The extracellular ATP changes were examined at microglia somata after the i.p. injection of LPS of 10 mg/kg (FIG. 8A). Similar to the GCaMP imaging experiment described above, the LPS injection induced a significant increase of GRAB fluorescence signals at one hour post-injection (FIGS. 8D and 8E). The fluorescence signals plateaued at two hours post-injection and remained at a high level at six hours post-injection (FIGS. 8D and 8E). The results show that the sustained increase of ATP signals at microglia somata after LPS injection differs from the LPS-induced ATP flashes observed in astrocytes, indicating cell type-specific ATP sensing following peripheral endotoxin challenge. No significant signal change was observed after the i.p. injection of saline (FIGS. 8D and 8E), again indicating that the elevated GRAB fluorescence signals were not due to the i.p. injection procedure.

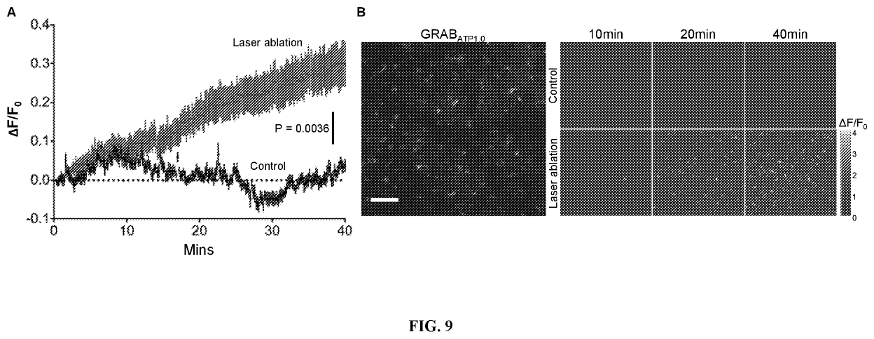

The extracellular ATP changes at microglia somata were also examined upon acute tissue damage. A brief laser ablation was applied to induce a local tissue damage, leading to an immediate increase in GRAB fluorescence signals that continued to ramp up for up to 40 min (FIG. 9).

### Example 8. Analysis of the Capability of AAV-MGs for Microglia Genome Editing

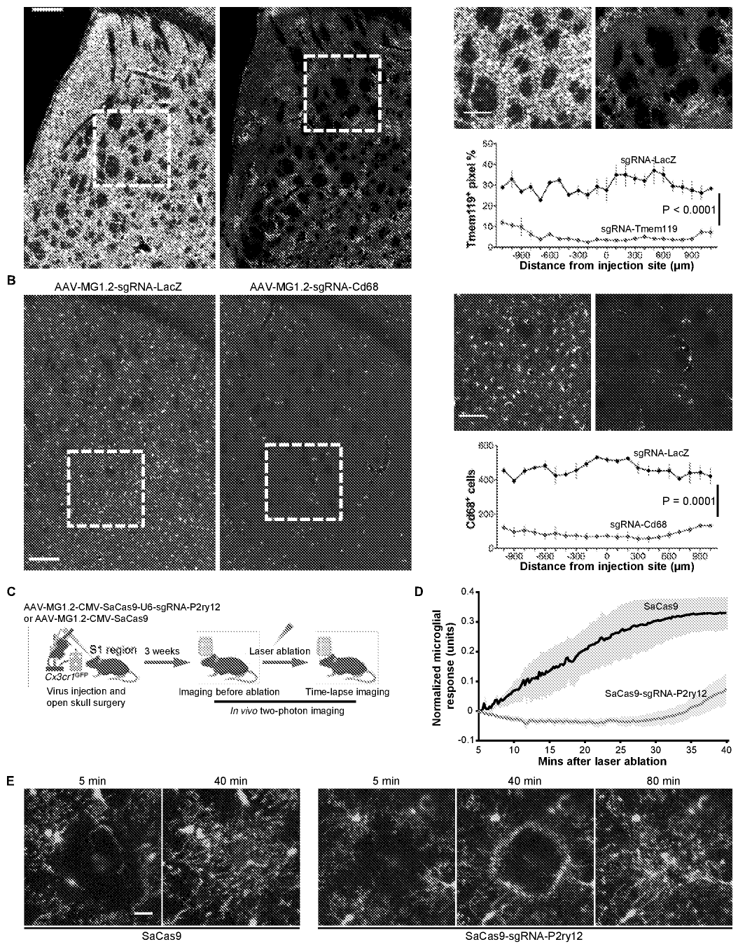

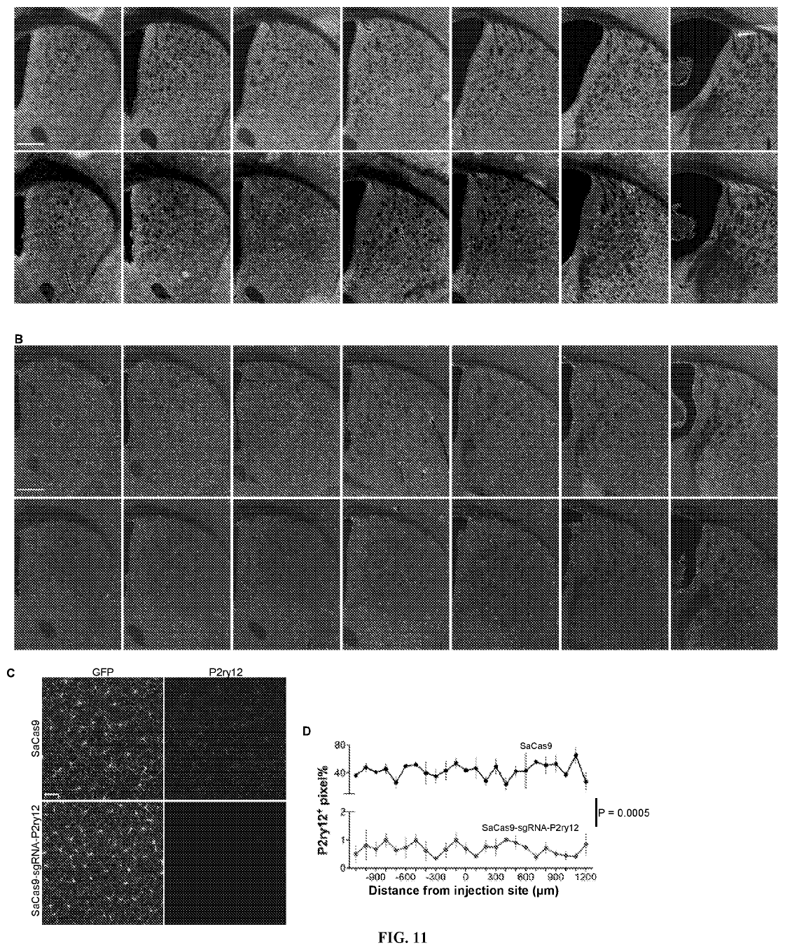

This example explored the utility of AAV-MGs for microglia genome editing. The Cre-dependent Rosa26 Cas9 reporter mouse (Rosa26-LSL-Cas9) was crossed with the Cx3cr1^{CreER} mouse. The expression of Streptococcus pyogenes Cas9 (SpCas9) in microglia was induced by tamoxifen i.p. injection. The AAV-MGs were used to package a single-stranded AAV vector that expresses a sgRNA. Two genes, Tmem119 and Cd68, that are selectively expressed in microglia in the brain were chosen as the targets for editing (AAV-MG1.1-sgRNA-Tmem119 and AAV-MG1.2-sgRNA-Cd68). The rAAVs harbored a sgRNA targeting LacZ served as the control virus (AAV-MG1.1/1.2-sgRNA-LacZ). Four weeks after injecting the rAAVs into the striatum, the knockout efficiency was examined by immunostaining. For both the target genes, the immunofluorescent signals of their encoded proteins were significantly reduced in the striatum in the mouse brains injected with the knockout rAAVs but not in the mouse brains injected with the control rAAVs (FIGS. 10A, 10B, 11A and 11B). Effective knockout of target genes was achieved over a large area in the dorsal striatum along the anterior-posterior axis by a single intrastriatal AAV injection (FIGS. 10A, 10B, 11A and 11B). These results show that combining the Cas9 transgenic mouse with AAV-MGs-mediated sgRNA delivery enables in vivo genome editing of microglia in a region-specific manner.

### Example 9. Demonstration of the Capability of AAV-MGs for Direct Microglia Genome Editing In Vivo

To further demonstrate direct microglia genome editing in vivo, AAV-MG1.2 was used to express a miniaturized Cas9 (Staphylococcus aureus Cas9, SaCas9) and a sgRNA from a single AAV vector (AAV-MG1.2-CMV-SaCas9-U6-sgRNA) in microglia. The sgRNA was designed for targeting the microglia homeostatic marker gene P2ry12 which is essential for microglial activation by extracellular polynucleotides. As shown by the depleted immunostaining signals, the gene P2ry12 was knocked out over a large area with a single injection of this rAAV in the Si cortex of Cx3cr1^{GFP} transgenic mice which selectively expresses GFP in microglia (FIGS. 11C and 11D). It has been known that the microglia immune activation can downregulate the expression of P2ry12. Control rAAV (AAV-MG1.2-CMV-SaCas9) that bears only the SaCas9 but not the sgRNA, was used in order to exclude the possibility that the observed P2ry12 knockout was due to rAAV-triggered microglial activation. The results show that expressing the SaCas9 without a sgRNA did not affect endogenous P2ry12 expression (FIGS. 11C and 11D), demonstrating that AAV-MG1.2-mediated transduction does not induce microglia activation and that the gene knockout by SaCas9 requires a specific sgRNA.

### Example 10. Examination of the Physiological Consequences of AAV-MG1.2-Mediated P2ry12-Knockout

In order to examine the physiological consequences of AAV-MG1.2-mediated P2ry12-knockout, the AAV-MG1.2-CMV-SaCas9-U6-sgRNA-P2ry12 or AAV-MG1.2-CMV-SaCas9 vector was injected into the S1 cortex of the Cx3cr1GFP transgenic mouse. Four weeks later, the microglial morphological responses to the tissue damage were tracked by means of in vivo two-photon imaging over the S1 cortex (FIG. 10C). Focal laser ablation in the mouse brains injected with the control rAAV induced immediate extension and recruitment of microglial processes towards the damage site, which reached the peak magnitude in 30-40 min after the ablation (FIGS. 10D and 10E). In stark contrast, the chemotactic responses of the microglia from those mouse brains injected with the P2ry12-knockout rAAV were dramatically reduced: microglial responses started only after ˜30 min and reached the peak magnitude after ˜80 min following focal laser ablation (FIGS. 10D and 10E). The AAV-based gene knockout approach circumvents the potential confounding effects of P2ry12 deficiency during development, and thus these results further support a role of P2ry12 in guiding the directional branch extension of microglia toward the sites of cortical damage.

### Example 11. Further Screen of a Semi-Randomly Mutating Capsid Library from AAV-cMG.QRP

To further improve the transduction efficiency of AAV-cMG.QRP in cultured mouse microglia, an additional round of directed evolution was conducted on AAV-cMG.QRP (FIG. 2D). First, an additional capsid library was generated by semi-randomly mutating the inserted heptamer and the adjacent four amino acids in AAV-cMG.QRP (FIG. 2D). This new library was packaged into rAAVs and screened in cultured mouse microglia. After one round of screening, a highly enriched capsid variant was identified and contained mutations at amino acid positions 594-596 of AAV-cMG.QRP (FIG. 2D). The variant comprises the amino acid sequence “PAD” at positions 594-596 and accounts for 0.79% of the total recovered variants (FIG. 2D). This variant was named as AAV-cMG. Significantly higher transduction rate and stronger mScarlet expression was achieved by AAV-cMG compared with that by the AAV5, AAV6TM, AAV8, AAV9, or AAV-cMG.QRP capsid (FIGS. 2E, 2F, and 2G). Similarly, doxorubicin also significantly enhanced the mSacrlet expression level of AAV-cMG in cultured mouse microglia (FIGS. 2H and 2I). The VP1 protein of AAV-cMG has an amino acid sequence as shown by SEQ ID NO.: 9.

The AAV-cMG.QRP mutant library was also screened in the brains of Cx3cr1^{CreER} mice. Two variants that are capable of transducing microglia in vivo were identified. The first variant, AAV-MG.TAF, comprises the amino acid sequence “TAF” at positions 589-591 of AAV-cMG.QRP (FIG. 12A). The second variant, AAV-MG.APA, comprises the amino acid sequence “APA” at positions 587-589 of AAV-cMG.QRP (FIG. 12B). The VP1 protein of AAV-MG.TAF has an amino acid sequence as shown by SEQ ID NO.: 53, and the VP1 protein of AAV-MG.APA has an amino acid sequence as shown by SEQ ID NO.: 56.

What is claimed is:

1. A recombinant adeno-associated virus (rAAV) capsid protein, comprising an amino acid sequence of 11 contiguous amino acids X₁X₂X₃X₄X₅X₆X₇X₈X₉X₁₀Q, wherein
X₁ is selected from Ala or Leu;
X₂ is selected from Gln, Met, Thr, Val or Pro;
X₃ is selected from Trp, Thr, Glu, Pro, Leu, Ala or Gln;
X₄ is selected from Pro, Thr, Met, Ser, Arg or Ala;
X₅ is selected from Pro, Ser, Val, Asp or Phe;
X₆ is selected from Lys or Pro;
X₇ is selected from Thr or Arg;
X₈ is selected from Thr, Glu or Pro;
X₉ is selected from Ser, Pro or Ala; and
X₁₀ is selected from Ala or Asp.

2. The rAAV capsid protein according to claim 1, wherein X₆ is Lys; X₇ is Thr; X₈ is Thr; X₉ is Ser; and/or X₁₀ is Ala.

3. The rAAV capsid protein according to claim 2, wherein X₁ is selected from Ala or Leu; X₂ is selected from Gln, Met, Thr or Val; X₃ is selected from Trp, Thr, Glu, Pro or Leu; X₄ is selected from Pro, Thr, Met or Ser; and/or X₅ is selected from Ser, Val, Asp or Pro.

4. The rAAV capsid protein according to claim 2, wherein
X₁ is Ala, X₂ is Gln, X₃ is Trp, X₄ is Pro, and X₅ is Pro;
X₁ is Leu, X₂ is Met, X₃ is Thr, X₄ is Pro, and X₅ is Pro;
X₁ is Ala, X₂ is Thr; X₃ is Glu, X₄ is Pro, and X₅ is Pro;
X₁ is Ala, X₂ is Gln; X₃ is Pro, X₄ is Thr, and X₅ is Ser;
X₁ is Ala, X₂ is Gln; X₃ is Leu, X₄ is Met, and X₅ is Val;
X₁ is Ala, X₂ is Gln; X₃ is Trp, X₄ is Thr, and X₅ is Asp; or
X₁ is Ala, X₂ is Val; X₃ is Leu, X₄ is Ser, and X₅ is Pro.

5. The rAAV capsid protein according to claim 1, wherein the capsid protein comprises an amino acid sequence selected from a group consisting of AQWPPKTTSAQ (SEQ ID NO.: 1), LMTPPKTTSAQ (SEQ ID NO.: 2), ATEPPKTTSAQ (SEQ ID NO.: 3), AQPTSKTTSAQ (SEQ ID NO.: 71), AQLMVKTTSAQ (SEQ ID NO.: 72), AQWTDKTTSAQ (SEQ ID NO.: 73), and AVLSPKTTSAQ (SEQ ID NO.: 74).

6. The rAAV capsid protein according to claim 1, wherein X₁ is Ala; X₆ is Pro; and/or X₇ is Arg.

7. The rAAV capsid protein according to claim 6, wherein X₂ is selected from Gln or Pro; X₃ is selected from Thr, Ala or Gln; X₄ is selected from Arg or Ala; X₅ is selected from Pro or Phe; X₈ is selected from Glu or Pro; X₉ is selected from Pro or Ala; and/or X₁₀ is selected from Ala or Asp.

8. The rAAV capsid protein according to claim 6, wherein
X₂ is Gln, X₃ is Gln, X₄ is Arg, X₅ is Pro, X₈ is Glu, X₉ is Pro, and X₁₀ is Ala;
X₂ is Gln, X₃ is Gln, X₄ is Arg, X₅ is Pro, X₈ is Pro, X₉ is Ala, and X₁₀ is Asp;
X₂ is Gln, X₃ is Thr, X₄ is Ala, X₅ is Phe, X₈ is Glu, X₉ is Pro, and X₁₀ is Ala; or
X₂ is Pro, X₃ is Ala, X₄ is Arg, X₅ is Pro, X₈ is Glu, X₉ is Pro, and X₁₀ is Ala.

9. The rAAV capsid protein according to claim 6, wherein the capsid protein comprises an amino acid sequence selected from a group consisting of AQQRPPREPAQ (SEQ ID NO.: 4), AQQRPPRPADQ (SEQ ID NO.: 5), AQTAFPREPAQ (SEQ ID NO.: 75) and APARPPREPAQ (SEQ ID NO.: 76).

10. The rAAV capsid protein according to claim 1, wherein the amino acid sequence of 11 contiguous amino acids X₁X₂X₃X₄X₅X₆X₇X₈X₉X₁₀Q is inserted in a GH-loop as compared to a wide-type capsid protein thereof, preferably inserted between amino acids 588 and 589 of the wide-type VP1, amino acids 451 and 452 of the wide-type VP2 and/or amino acids 386 and 387 of the wide-type VP3, of AAV9 or the corresponding position in the capsid protein of another AAV serotype than AAV9.

11. A polynucleotide sequence encoding the rAAV capsid protein as defined in claim 1.

12. A recombinant adeno-associated virus (rAAV) vector comprising the capsid protein as defined in claim cl.

13. The rAAV vector according to claim 12, further comprising a heterologous polynucleotide sequence, which preferably encodes a heterologous polypeptide, a non-coding RNA or a CRISPR agent.

14. The rAAV vector according to claim 13, wherein the heterologous polynucleotide sequence encodes miRNA, siRNA, piRNA, lncRNA, or a guide RNA.

15. A pharmaceutical composition comprising the rAAV vector as defined in claim 12, and one or more pharmaceutically acceptable carriers.

16. A method for delivering the rAAV vector as defined in claim 12, comprising contacting the target cell with the rAAV vector.

17. The method according to claim 16, wherein the target cell is microglia in vitro or in vivo, preferably in the presence of an inhibitor for a topoisomeras, proteasome or nonhomologous-end joining, or a DNA damage inducer, wherein the inhibitor more preferably is selected from a group consisting of doxorubicin, bortezomib, etoposide, teniposide, vanillin and bleomycin.

18. A host cell comprising the rAAV vector as defined in claim 12.

19. A method for treating a neurological disorder, comprising administering a therapeutically effective amount of the pharmaceutical composition of claim 15 to a subject in need thereof, preferably the neurological disorder is a disease associated with microglia.

20. The method according to claim 19, wherein the neurological disorder comprises Alzheimer's disease, Parkinson's disease, Huntington's disease, amyotrophic lateral sclerosis, leukoencephalopathy, glioma and atypical teratoid/rhabdoid tumor.
